(19) United States
(12) Patent Application Publication
(10) Pub. No.: US 2009/0069376 A1
(43) Pub. Date: Mar. 12, 2009

(54) VLA-4 Antagonists

(76) Inventors: Ping Liu, Westfield, NJ (US); Carrie Jones, Cambridge, MA (US); Thomas S. Reger, Lansdale, PA (US)

Correspondence Address: MERCK AND CO., INC, P O BOX 2000, RAHWAY, NJ, 07065-0907, US

(21) Appl. No.: 11/887,701

(22) PCT Filed: Apr. 17, 2006
(86) PCT No.: PCT/US2006/014655

§ 371 Date: Oct. 2, 2007

Related U.S. Application Data
(60) Provisional application No. 60/673,418, filed on Apr. 21, 2005.

(51) Int. Cl. A61K 31/4545; C07D 401/12; A61K 31/4439; A61P 11/06; A61P 35/00; A61P 35/02; A61P 19/02; A61P 9/00; A61P 1/00

(52) U.S. Cl. 514/318; 546/278.4; 546/277.7; 546/194; 514/339; 514/343

(57) ABSTRACT

Substituted N—[N-(sulphonylphenyl)sulfonyl-prolyl]-phenylalanine derivatives of the present invention are antagonists of the VLA-4 integrin and are useful in the treatment, prevention and suppression of diseases mediated by VLA-4-binding and cell adhesion and activation. Moreover, the compounds of the present invention demonstrate significant receptor occupancy of VLA-4 bearing cells after oral administration and are suitable for once-, twice-, or thrice-a-day oral administration. This invention also relates to compositions containing such compounds and methods of treatment using such compounds.

### BACKGROUND OF THE INVENTION

VLA-4 (“very late antigen-4”; CD49d/CD29; or α₄β₁) is an integrin expressed on all leukocytes, except platelets and mature neutrophils, including dendritic cells and macrophage-like cells and is a key mediator of the cell-cell and cell-matrix interactions of these cell types. The ligands for VLA-4 include vascular cell adhesion molecule-1 (VCAM-1), the CS-1 domain of fibronectin (FN), and the matrix protein, osteopontin. Neutralizing anti-α₄ antibodies or blocking peptides that inhibit the interaction between VLA-4 and its ligands have been shown to be efficacious both prophylactically and therapeutically in several animal models of disease including asthma, multiple sclerosis, inflammatory bowel disease, multiple myeloma, and rheumatoid arthritis.

The humanized monoclonal antibody against α₄, natalizumab (Tysabri®, Elan/Biogen), has demonstrated efficacy in the treatment of multiple sclerosis (D. H. Miller et al., New England Journal of Medicine, 348, 15 (2003)) and Crohn's disease (S. Ghosh et al. New England Journal of Medicine, 348, 23 (2003)). There are also several VLA-4 antagonists in early clinical trials for treatment of asthma, arthritis, multiple sclerosis, and Crohn's disease.

In the early clinical trials with natalizumab, lymphocytosis (a surrogate marker for blockade of VLA-4 function) and >80% receptor occupancy were observed. A small molecule VLA-4 antagonist was reported to demonstrate functional activity in the rat experimental autoimmune encephalomyelitis (EAE) assay, an animal model of multiple sclerosis following subcutaneous administration (D. R. Leone et al., J. Pharmacol. Exper. Therap., 305, 1150 (2003). This compound was shown to induce lymphocytosis, and to have a slow dissociation rate (off-rate) resulting in significant and sustained receptor occupancy on VLA-4-bearing cells. There was a positive correlation between receptor occupancy, lymphocytosis, and efficacy in the EAE model described in this manuscript.

A series of isonicotinoyl-L-aminophenylalanine derivatives shown to possess slow dissociation (off-rate) from VLA-4 on Jurkat cells were reported in G. Doherty et al., Bioorganic & Medicinal Chemistry Letters, 13, 1891 (2003). However, the compound that was further characterized demonstrated very poor pharmacokinetic properties such as low oral bioavailability, moderate to high plasma clearance and a short half-life rendering it unsuitable for oral administration. Compounds of the present invention are potent antagonists of VLA-4 capable of achieving and maintaining receptor occupancy for a time sufficient to allow for oral administration.

### SUMMARY OF THE INVENTION

Substituted N—[N-(sulphonylphenyl)sulfonyl-prolyl]-phenylalanine derivatives of the present invention are antagonists of the VLA-4 integrin and are useful in the treatment, prevention and suppression of diseases mediated by VLA-4-binding and cell adhesion and activation. Moreover, the compounds of the present invention demonstrate significant receptor occupancy of VLA-4 bearing cells after oral administration and are suitable for once-, twice-, or thrice-a-day oral administration. This invention also relates to compositions containing such compounds and methods of treatment using such compounds.

### DETAILED DESCRIPTION OF THE INVENTION

The present invention provides compounds of Formula I:

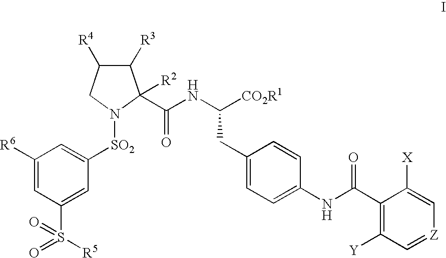

or a pharmaceutically acceptable salt thereof, wherein:
X and Y are independently chosen from (1) C₁₋₃alkyl, (2) halogen, and (3) C₁₋₃alkoxy;

### Z is N or N⁺O⁻;

R¹ is selected from (1) hydrogen, (2) C₁₋₁₀alkyl, (3) —(C₁₋₁₀alkyl)-aryl, (4) —(C₁₋₁₀alkyl)-O—C₁₋₁₀alkyl, (5) —(C₁₋₁₀alkyl)-OC(O)—C₁₋₁₀ alkyl, (6) —(C₁₋₁₀alkyl)-OC(O)-aryl, and (7) —(C₁₋₁₀alkyl)-OC(O)O—C₁₋₁₀alkyl; wherein alkyl is optionally substituted with one to three substituents independently selected from R^{a} and aryl is optionally substituted with one to three substituents independently selected from Rb;
R² is hydrogen or methyl;
R³ and R⁴ are independently selected from (1) hydrogen, (2) C₁₋₁₀alkyl, (3) —OR^{d}, (4) —NR^{d}R^{e}, (5) —NR^{d}S(O)ₘR^{e}, (6) —NR^{d}C(O)R^{e}, (7) —NR^{d}C(O)OR^{e} and (8) —NR^{d}C(O)NR^{d}R^{e}, wherein alkyl is optionally substituted with one to four substituents independently selected from R^{a}; or
R³ and R⁴ together with the carbon atoms to which they are attached form a monocyclic ring of 5 to 7 members containing 0-2 additional heteroatoms independently selected from O, S and N—R^{h}, said ring optionally substituted with one to four substituents independently selected from R^{c};
R⁵ is selected from (1) C₁₋₁₀alkyl, and (2) aryl;
R⁶ is selected from (1) hydrogen, (2) halogen, and (3) —OR^{d};
R^{a} is selected from (1) —OR^{d}, (2) —NR^{d}S(O)ₘR^{e}, (3) —NO₂, (4) halogen, (5) —S(O)ₘR^{d}, (6) —SR^{d}, (7) —S(O)₂OR^{d}, (8) —S(O)ₘNR^{d}R^{e}, (9) —NR^{d}R^{e}, (10) —O(CR^{f}R^{g})ₙNR^{d}R^{e}, (11) —C(O)R^{d}, (12) —CO₂R^{d}, (13) —CO₂(CR^{f}R^{g})ₙCONR^{d}R^{e}, (14) —OC(O)R^{d}, (15) —CN, (16) —C(O)NR^{d}R^{e}, (17) —NR^{d}C(O)R^{e}, (18) —OC(O)NR^{d}R^{e}, (19) —NR^{d}C(O)OR^{e}, (20) —NR^{d}C(O)NR^{d}R^{e}, (21) —CR^{d}(N—OR^{e}), (22) CF₃, (23) —OCF₃, (24) C₃₋₈cycloalkyl, and (25) heterocyclyl; wherein cycloalkyl and heterocyclyl are optionally substituted with one to three groups independently selected from R^{c};
R^{b} is selected from (1) a group selected from R^{a}, (2) C₁₋₁₀ alkyl, (3) C₂₋₁₀ alkenyl (4) C₂₋₁₀ alkynyl, (5) aryl, and (6) —(C₁₋₁₀alkyl)-aryl, wherein alkyl, alkenyl, alkynyl, and aryl are optionally substituted with one to three substituents selected from a group independently selected from R^{c};
R^{c} is (1) halogen, (2) amino, (3) carboxy, (4) C₁₋₄alkyl, (5) C₁₋₄alkoxy, (6) aryl, (7) —(C₁₋₄alkyl)-aryl, (8) hydroxy, (9) CF₃, (10) OC(O)C₁₋₄alkyl, (11) —CN, and (12) —SO₂C₁₋₁₀alkyl;
R^{d} and R^{e} are independently selected from hydrogen, C₁₋₁₀ alkyl, C₂₋₁₀alkenyl, C₂₋₁₀alkynyl, Cy and Cy-C₁₋₁₀alkyl, wherein alkyl, alkenyl, alkynyl and Cy are optionally substituted with one to four substituents independently selected from R^{c}; or
R^{d} and R^{e} together with the atom(s) to which they are attached form a heterocyclic ring of 4 to 7 members containing 0-2 additional heteroatoms independently selected from O, S and N—R^{h}; wherein said ring is optionally substituted with one to four substituents independently selected from R^{c};
R^{f} and R^{g} are independently selected from hydrogen, C₁₋₁₀alkyl, Cy and Cy-C₁₋₁₀alkyl; or
R^{f} and R^{g} together with the carbon to which they are attached form a ring of 5 to 7 members containing 0-2 heteroatoms independently selected from oxygen, sulfur and nitrogen;
R^{h} is selected from R^{f} and —C(O)R^{f};
Cy is selected from cycloalkyl, heterocyclyl, aryl, and heteroaryl;
each m is independently 0, 1 or 2; and
each n is independently 1, 2, 3, or 4.

In one embodiment of formula I, one of X and Y is halogen and the other is selected from halogen, C₁₋₃alkyl and C₁₋₃alkoxy. In one subset of this embodiment, one of X and Y is chloro and the other is chloro or methoxy. In another subset X and Y are each chloro.

In another embodiment of formula I, R¹ is hydrogen, C₁₋₄alkyl, —(C₁₋₄alkyl)OC(O)—C₁₋₄alkyl, or —(C₁₋₄alkyl)OC(O)—C₁₋₄alkyl. In one subset R¹ is hydrogen, and in another subset R¹ is C₁₋₄alkyl.

In another embodiment of formula I, R³ is hydrogen, and R⁴ is NR^{d}R^{e}.

In another embodiment of formula I, R³ is NR^{d}R^{e} and R⁴ is hydrogen.

One embodiment of formula I provides compounds of formula Ia:

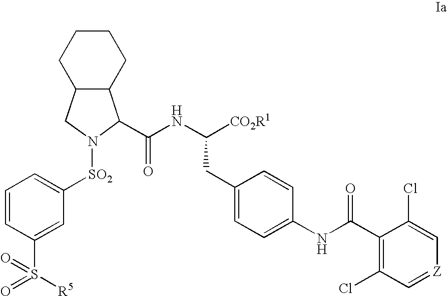

or a pharmaceutically acceptable salt thereof, wherein

### Z is N or N⁺O⁻;

R¹ is selected from hydrogen, C₁₋₁₀alkyl, —(C₁₋₄alkyl)-aryl, —(C₁₋₄alkyl)-O—C₁₋₄alkyl, and —(C₁₋₄alkyl)-OC(O)—C₁₋₄alkyl;
R⁵ is selected from C₁₋₄alkyl and phenyl.

Representative compounds of formula I are as follows:

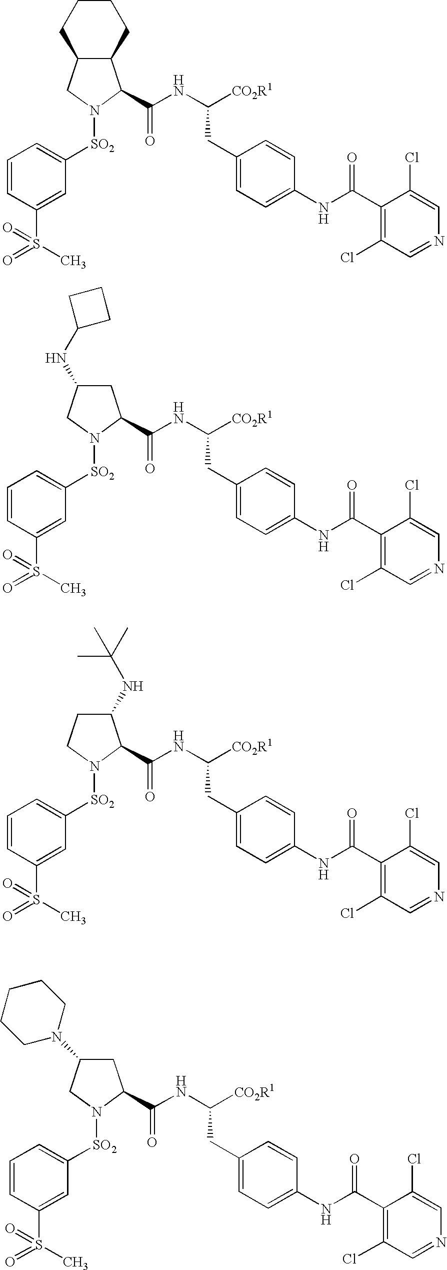

where R¹ is H or ethyl, and pharmaceutically acceptable salts thereof.

Another embodiment of Formula I provides compounds of Formula Ib:

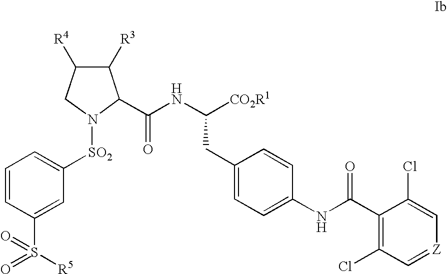

or a pharmaceutically acceptable salt thereof, wherein

### Z is N or N⁺O⁻;

R¹ is selected hydrogen and C₁₋₄alkyl;
R⁵ is selected from C₁₋₄alkyl and phenyl; and
R³ is hydrogen and R⁴ is NR^{d}R^{e} or R³ is NR^{d}R^{e} and R⁴ is hydrogen. In a subset of this embodiment, one of R³ or R⁴ is hydrogen and the other of R³ or R⁴ is selected from the group consisting of: C₁₋₆alkylamino, C₃₋₆cycloalkylamino and

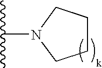

wherein k is 0 to 3. Within this subset, one of R³ or R⁴ is hydrogen and the other of R³ or R⁴ is selected from the group consisting of: cyclobutylamino, tert-butylamino and piperidino.

In another aspect the present invention provides a method for the prevention or treatment of diseases, disorders, conditions or symptoms mediated by cell adhesion in a mammal which comprises administering to said mammal an effective amount of a compound of formula I. This aspect includes the use of a compound of formula I in the manufacture of a medicament for the treatment of diseases, disorders, conditions or symptoms mediated by cell adhesion in a mammal. In one embodiment said disease or disorder is selected from asthma, allergic rhinitis, chronic obstructory pulmonary disease (COPD), multiple sclerosis, atherosclerosis, inflammatory bowel disease, rheumatoid arthritis, organ transplantation, acute leukemia, and sickle cell anemia.

In another aspect the present invention provides a method for preventing the action of VLA-4 in a mammal which comprises administering to said mammal a therapeutically effective amount of a compound of formula I.

Another aspect of the present invention provides a pharmaceutical composition which comprises a compound of formula I and a pharmaceutically acceptable carrier.

“Alkyl”, as well as other groups having the prefix “alk”, such as alkoxy, alkanoyl, means carbon chains which may be linear or branched or combinations thereof. Examples of alkyl groups include methyl, ethyl, propyl, isopropyl, butyl, sec- and tert-butyl, pentyl, hexyl, heptyl, octyl, nonyl, and the like.

“Alkenyl” means carbon chains which contain at least one carbon-carbon double bond, and which may be linear or branched or combinations thereof. Examples of alkenyl include vinyl, allyl, isopropenyl, pentenyl, hexenyl, heptenyl, 1-propenyl, 2-butenyl, 2-methyl-2-butenyl, and the like.

“Alkynyl” means carbon chains which contain at least one carbon-carbon triple bond, and which may be linear or branched or combinations thereof. Examples of alkynyl include ethynyl, propargyl, 3-methyl-1-pentynyl, 2-heptynyl and the like.

“Cycloalkyl” means mono- or bicyclic saturated carbocyclic rings, each of which having from 3 to 10 carbon atoms. The term also includes monocyclic rings fused to an aryl group in which the point of attachment is on the non-aromatic portion. Examples of cycloalkyl include cyclopropyl, cyclobutyl, cyclopentyl, cyclohexyl, cycloheptyl, tetrahydronaphthyl, decahydronaphthyl, indanyl, and the like.

“Aryl” means mono- or bicyclic aromatic rings containing only carbon atoms. The term also includes aryl group fused to a monocyclic cycloalkyl or monocyclic heterocyclyl group in which the point of attachment is on the aromatic portion. Examples of aryl include phenyl, naphthyl, indanyl, indenyl, tetrahydronaphthyl, 2,3-dihydrobenzofuranyl, dihydrobenzopyranyl, 1,4-benzodioxanyl, and the like.

“Heteroaryl” means a mono- or bicyclic aromatic ring containing at least one heteroatom selected from N, O and S, with each ring containing 5 to 6 atoms. Examples of heteroaryl include pyrrolyl, isoxazolyl, isothiazolyl, pyrazolyl, pyridyl, oxazolyl, oxadiazolyl, thiadiazolyl, thiazolyl, imidazolyl, triazolyl, tetrazolyl, furanyl, triazinyl, thienyl, pyrimidyl, pyridazinyl, pyrazinyl, benzoxazolyl, benzothiazolyl, benzimidazolyl, benzofuranyl, benzothiophenyl, furo(2,3-b)pyridyl, quinolyl, indolyl, isoquinolyl, and the like.

“Heterocyclyl” means mono- or bicyclic saturated rings containing at least one heteroatom selected from N, S and O, each of said ring having from 3 to 10 atoms in which the point of attachment may be carbon or nitrogen. The term also includes monocyclic heterocycle fused to an aryl or heteroaryl group in which the point of attachment is on the non-aromatic portion. Examples of “heterocyclyl” include pyrrolidinyl, piperidinyl, piperazinyl, imidazolidinyl, 2,3-dihydrofuro(2,3-b)pyridyl, benzoxazinyl, tetrahydrohydroquinolinyl, tetrahydroisoquinolinyl, dihydroindolyl, and the like. The term also includes partially unsaturated monocyclic rings that are not aromatic, such as 2- or 4-pyridones attached through the nitrogen or N-substituted-(1H,3H)-pyrimidine-2,4-diones (N-substituted uracils).

“Halogen” includes fluorine, chlorine, bromine and iodine.

### Optical Isomers—Diastereomers—Geometric Isomers—Tautomers

Compounds of Formula I contain one or more asymmetric centers and can thus occur as racemates and racemic mixtures, single enantiomers, diastereomeric mixtures and individual diastereomers. The present invention is meant to comprehend all such isomeric forms of the compounds of Formula I.

Some of the compounds described herein contain olefinic double bonds, and unless specified otherwise, are meant to include both E and Z geometric isomers.

Some of the compounds described herein may exist with different points of attachment of hydrogen, referred to as tautomers. Such an example may be a ketone and its enol form known as keto-enol tautomers. The individual tautomers as well as mixture thereof are encompassed with compounds of Formula I.

Compounds of the Formula I may be separated into diastereoisomeric pairs of enantiomers by, for example, fractional crystallization from a suitable solvent, for example MeOH or EtOAc or a mixture thereof. The pair of enantiomers thus obtained may be separated into individual stereoisomers by conventional means, for example by the use of an optically active amine as a resolving agent or on a chiral HPLC column.

Alternatively, any enantiomer of a compound of the general Formula I or Ia may be obtained by stereospecific synthesis using optically pure starting materials or reagents of known configuration.

### Salts

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, and tartaric acids.

It will be understood that, as used herein, references to the compounds of Formula I are meant to also include the pharmaceutically acceptable salts.

### Utilities

The ability of the compounds of Formula I to antagonize the actions of VLA-4 integrin makes them useful for preventing or reversing the symptoms, disorders or diseases induced by the binding of VLA-4 to its various ligands. Thus, these antagonists will inhibit cell adhesion processes including cell activation, migration, proliferation and differentiation. Accordingly, another aspect of the present invention provides a method for the treatment (including prevention, alleviation, amelioration or suppression) of diseases or disorders or symptoms mediated by VLA-4 binding and cell adhesion and activation, which comprises administering to a mammal an effective amount of a compound of Formula I. Such diseases, disorders, conditions or symptoms are, for example (1) multiple sclerosis, (2) asthma, (3) allergic rhinitis, (4) allergic conjunctivitis, (5) inflammatory lung diseases, (6) rheumatoid arthritis, (7) septic arthritis, (8) type I diabetes, (9) organ transplantation rejection, (10) restenosis, (11) autologous bone marrow transplantation, (12) inflammatory sequelae of viral infections, (13) myocarditis, (14) inflammatory bowel disease including ulcerative colitis and Crohn's disease, (15) certain types of toxic and immune-based nephritis, (16) contact dermal hypersensitivity, (17) psoriasis, (18) tumor metastasis, (19) atherosclerosis, (20) sickle cell anemia, (21) certain acute leukemias, (22) various melanomas, carcinomas and sarcomas (including multiple myeloma); (23) acute respiratory distress syndrome; (24) uveitis; (25) circulatory shock; (26) hepatitis, and (27) chronic obstructive pulmonary disease. The compounds of the present invention may be useful for the treatment of the above-recited diseases, disorders, conditions or symptoms in mammals other than humans, including, for example, horses, cats, dogs, cows and pigs. The instant compounds may also be useful for the treatment of allergy-related or allergy-induced respiratory conditions in nonhuman mammals, including the treatment of recurrent airway obstruction, commonly called heaves, in horses.

The utilities of the present compounds in these diseases or disorders may be demonstrated in animal disease models that have been reported in the literature. The following are examples of such animal disease models: i) experimental allergic encephalomyelitis, a model of neuronal demyelination resembling multiple sclerosis (for example, see T. Yednock et al., Nature, 356, 63 (1993) and E. Keszthelyi et al., Neurology, 47, 1053 (1996)); ii) bronchial hyperresponsiveness in sheep and guinea pigs as models for the various phases of asthma (for example, see W. M. Abraham et al., J. Clin. Invest. 93, 776 (1993) and A. A. Y. Milne and P. P. Piper, Eur. J. Pharmacol., 282, 243 (1995)); iii) adjuvant-induced arthritis in rats as a model of inflammatory arthritis (see C. Barbadillo et al., Arthr. Rheuma. (Suppl.), 36 95 (1993) and D. Seiffge, J. Rheumatol., 23, 12 (1996)); iv) adoptive autoimmune diabetes in the NOD mouse (see J. L. Baron et al., J. Clin. Invest., 93, 1700 (1994), A. Jakubowski et al., J. Immunol., 155, 938 (1995), and X. D. Yang et al., Diabetes, 46, 1542 (1997)); v) cardiac allograft survival in mice as a model of organ transplantation (see M. Isobe et al., Tranplant. Proc., 26, 867 (1994) and S. Molossi et al., J. Clin Invest., 95, 2601 (1995)); vi) spontaneous chronic colitis in cotton-top tamarins which resembles human ulcerative colitis, a form of inflammatory bowel disease (see D. K. Podolsky et al., J. Clin. Invest., 92, 372 (1993)); vii) contact hypersensitivity models as a model for skin allergic reactions (see T. A. Ferguson and T. S. Kupper, J. Immunol., 150, 1172 (1993) and P. L. Chisholm et al., Eur. J. Immunol., 23, 682 (1993)); viii) acute nephrotoxic nephritis (see M. S. Mulligan et al., J. Clin. Invest., 91, 577 (1993)); ix) tumor metastasis (for examples, see M. Edward, Curr. Opin. Oncol., 7, 185 (1995)); x) experimental autoimmune thyroiditis (see R. W. McMurray et al., Autoimmunity, 23, 9 (1996); xi) ischemic tissue damage following arterial occlusion in rats (see F. Squadrito et al., Eur. J. Pharmacol., 318, 153 (1996)); xii) inhibition of TH2 T-cell cytokine production including IL-4 and IL-5 by VLA-4 antibodies which would attenuate allergic responses (J. Clinical Investigation 100, 3083 (1997); xiii) antibodies to VLA-4 integrin mobilize long term repopulating cells and augment cytokine-induced mobilization in primates and mice (Blood, 90 4779-4788 (1997); xiv) sickle reticulocytes adhere to VCAM-1 (Blood 85 268-274 (1995) and Blood 88 4348-4358 (1996); xv) chemokine stromal cell derived factor 1 modulates VLA-4 integrin mediated multiple myeloma cell adhesion to CS-1/fibronectin and VCAM-1 (Blood, 97, 346-351 2001); xvi) Anti-α4 integrin antibody suppresses the development of multiple myeloma and associated osteoclastic osteolysis (see Y. Mori et al., Blood, 104 2149-2154).

### Dose Ranges

The magnitude of prophylactic or therapeutic dose of a compound of Formula I will, of course, vary with the nature and severity of the condition to be treated, and with the particular compound of Formula I used and its route of administration. The dose will also vary according to the age, weight and response of the individual patient. In general, the daily dose range lie within the range of from about 0.001 mg to about 100 mg per kg body weight of a mammal, preferably 0.01 mg to about 50 mg per kg, and most preferably 0.1 to 10 mg per kg, in single or divided doses. On the other hand, it may be necessary to use dosages outside these limits in some cases.

For use where a composition for intravenous administration is employed, a suitable dosage range is from about 0.01 mg to about 25 mg (preferably from 0.1 mg to about 10 mg) of a compound of Formula I per kg of body weight per day.

In the case where an oral composition is employed, a suitable dosage range is, e.g. from about 0.01 mg to about 100 mg of a compound of Formula I per kg of body weight per day, preferably from about 0.1 mg to about 10 mg per kg.

For use where a composition for sublingual administration is employed, a suitable dosage range is from 0.01 mg to about 25 mg (preferably from 0.1 mg to about 5 mg) of a compound of Formula I per kg of body weight per day.

For the treatment of asthma, a compound of Formula I may be used at a dose of from about 0.1 mg/kg to about 100 mg/kg, preferably from about 1 mg/kg to 10 mg/kg, by oral/inhalation/sublingual/etc. once, twice, three times daily, etc. The dose may be adminstered as a single daily dose or divided for twice or thrice daily administration.

For the treatment of multiple sclerosis, a compound of Formula I may be used at a dose of from about 0.1 mg/kg to about 100 mg/kg, preferably from about 1 mg/kg to 10 mg/kg, by oral/inhalation/sublingual/etc. once, twice, three times daily, etc. The dose may be adminstered as a single daily dose or divided for twice or thrice daily administration.

For the treatment of inflammatory bowel disease, a compound of Formula I may be used at a dose of from about 0.1 mg/kg to about 100 mg/kg, preferably from about 1 mg/kg to 10 mg/kg, by oral/inhalation/etc. once, twice, three times daily, etc. The dose may be adminstered as a single daily dose or divided for twice or thrice daily administration.

For the treatment of rheumatoid arthritis, a compound of Formula I may be used at a dose of from about 0.1 mg/kg to about 100 mg/kg, preferably from about 1 mg/kg to 10 mg/kg, by oral/inhalation/sublingual/etc. once, twice, three times daily, etc. The dose may be adminstered as a single daily dose or divided for twice or thrice daily administration.

### Pharmaceutical Compositions

Another aspect of the present invention provides pharmaceutical compositions which comprises a compound of Formula I and a pharmaceutically acceptable carrier. The term “composition”, as in pharmaceutical composition, is intended to encompass a product comprising the active ingredient(s), and the inert ingredient(s) (pharmaceutically acceptable excipients) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of Formula I, additional active ingredient(s), and pharmaceutically acceptable excipients.

Any suitable route of administration may be employed for providing a mammal, especially a human with an effective dosage of a compound of the present invention. For example, oral, rectal, topical, parenteral, ocular, pulmonary, nasal, and the like may be employed. Dosage forms include tablets, troches, dispersions, suspensions, solutions, capsules, creams, ointments, aerosols, and the like.

The pharmaceutical compositions of the present invention comprise a compound of Formula I as an active ingredient or a pharmaceutically acceptable salt thereof, and may also contain a pharmaceutically acceptable carrier and optionally other therapeutic ingredients. The term “pharmaceutically acceptable salts” refers to salts prepared from pharmaceutically acceptable non-toxic bases or acids including inorganic bases or acids and organic bases or acids.

The compositions include compositions suitable for oral, sublingual, rectal, topical, parenteral (including subcutaneous, intramuscular, and intravenous), ocular (ophthalmic), pulmonary (aerosol inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

For administration by inhalation, the compounds of the present invention are conveniently delivered in the form of an aerosol spray presentation from pressurized packs or nebulizers. The compounds may also be delivered as powders which may be formulated and the powder composition may be inhaled with the aid of an insufflation powder inhaler device. The preferred delivery systems for inhalation are metered dose inhalation (MD) aerosol, which may be formulated as a suspension or solution of a compound of Formula I in suitable propellants, such as fluorocarbons or hydrocarbons and dry powder inhalation (DPI) aerosol, which may be formulated as a dry powder of a compound of Formula I with or without additional excipients.

Suitable topical formulations of a compound of formula I include transdermal devices, aerosols, creams, ointments, lotions, dusting powders, and the like.

In practical use, the compounds of Formula I can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, capsules and tablets, with the solid oral preparations being preferred over the liquid preparations. Because of their ease of administration, tablets and capsules represent the most advantageous oral dosage unit form in which case solid pharmaceutical carriers are obviously employed. If desired, tablets may be coated by standard aqueous or nonaqueous techniques.

In addition to the common dosage forms set out above, the compounds of Formula I may also be administered by controlled release means and/or delivery devices such as those described in U.S. Pat. Nos. 3,845,770; 3,916,899; 3,536,809; 3,598,123; 3,630,200 and 4,008,719.

Pharmaceutical compositions of the present invention suitable for oral administration may be presented as discrete units such as capsules, cachets or tablets each containing a predetermined amount of the active ingredient, as a powder or granules or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association the active ingredient with the carrier which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet may be prepared by compression or molding, optionally with one or more accessory ingredients. Compressed tablets may be prepared by compressing in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Desirably, each tablet contains from about 1 mg to about 500 mg of the active ingredient and each cachet or capsule contains from about 1 to about 500 mg of the active ingredient.

The following are examples of representative pharmaceutical dosage forms for the compounds of Formula I:

| Inj. Suspension (I.M.) | mg/mL | Tablet | mg/tab. | Capsule | mg/cap. |
|---|---|---|---|---|---|
| Cmpd of Formula I | 10 | Cmpd of Formula I | 25 | Cmpd of Formula I | 25 |
| Methylcellulose | 5.0 | Microcryst. Cellulose | 415 | Lactose Powder | 573.5 |
| Tween 80 | 0.5 | Povidone | 14.0 | Magnesium Stearate | 1.5 |
| Benzyl alcohol | 9.0 | Pregelatinized Starch | 43.5 | | 600 |
| Benzalkonium chloride | 1.0 | Magnesium Stearate | 2.5 | | |
| Water for injection to a total | | | 500 | | |
| volume of 1 mL | | | | | |

| Aerosol | Per canister | |
|---|---|---|
| Compound of Formula I | 24 | mg |
| Lecithin, NF Liq. Conc. | 1.2 | mg |
| Trichlorofluoromethane, NF | 4.025 | g |
| Dichlorodifluoromethane, NF | 12.15 | g |

### Combination Therapy

Compounds of Formula I may be used in combination with other drugs that are used in the treatment/prevention/suppression or amelioration of the diseases or conditions for which compounds of Formula I are useful. Such other drugs may be administered, by a route and in an amount commonly used therefor, contemporaneously or sequentially with a compound of Formula I. When a compound of Formula I is used contemporaneously with one or more other drugs, a pharmaceutical composition containing such other drugs in addition to the compound of Formula I is preferred. Accordingly, the pharmaceutical compositions of the present invention include those that also contain one or more other active ingredients, in addition to a compound of Formula I. Examples of other active ingredients that may be combined with a compound of Formula I, either administered separately or in the same pharmaceutical compositions, include, but are not limited to: (a) other VLA-4 antagonists such as those described in U.S. Pat. No. 5,510,332, WO97/03094, WO97/02289, WO96/40781, WO96/22966, WO96/20216, WO96/01644, WO96/06108, WO95/15973 and WO96/31206, as well as natalizumab; (b) steroids such as beclomethasone, methylprednisolone, betamethasone, prednisone, dexamethasone, and hydrocortisone; (c) immunosuppressants such as cyclosporin, tacrolimus, rapamycin and other FK-506 type immunosuppressants; (d) antihistamines (H1-histamine antagonists) such as bromopheniramine, chlorpheniramine, dexchlorpheniramine, triprolidine, clemastine, diphenhydramine, diphenylpyraline, tripelennamine, hydroxyzine, methdilazine, promethazine, trimeprazine, azatadine, cyproheptadine, antazoline, pheniramine pyrilamine, astemizole, terfenadine, loratadine, cetirizine, fexofenadine, descarboethoxyloratadine, and the like; (e) non-steroidal anti-asthmatics such as β2-agonists (terbutaline, metaproterenol, fenoterol, isoetharine, albuterol, bitolterol, salmeterol and pirbuterol), theophylline, cromolyn sodium, atropine, ipratropium bromide, leukotriene antagonists (zafirlukast, montelukast, pranlukast, iralukast, pobilukast, SKB-106,203), leukotriene biosynthesis inhibitors (zileuton, BAY-1005); (f) non-steroidal antiinflammatory agents (NSAIDs) such as propionic acid derivatives (alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid, and tioxaprofen), acetic acid derivatives (indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin, and zomepirac), fenamic acid derivatives (flufenamic acid, meclofenamic acid, mefenamic acid, niflumic acid and tolfenamic acid), biphenylcarboxylic acid derivatives (diflunisal and flufenisal), oxicams (isoxicam, piroxicam, sudoxicam and tenoxican), salicylates (acetyl salicylic acid, sulfasalazine) and the pyrazolones (apazone, bezpiperylon, feprazone, mofebutazone, oxyphenbutazone, phenylbutazone); (g) cyclooxygenase-2 (COX-2) inhibitors such as celecoxib, rofecoxib, and parecoxib; (h) inhibitors of phosphodiesterase type IV (PDE-IV); (i) antagonists of the chemokine receptors, especially CCR-1, CCR-2, and CCR-3; 0) cholesterol lowering agents such as HMG-CoA reductase inhibitors (lovastatin, simvastatin, pravastatin, fluvastatin, atorvastatin, and other statins), sequestrants (cholestyramine and colestipol), nicotinic acid, fenofibric acid derivatives (gemfibrozil, clofibrat, fenofibrate and benzafibrate), and probucol; (k) anti-diabetic agents such as insulin, sulfonylureas, biguanides (metformin), a-glucosidase inhibitors (acarbose) and glitazones (troglitazone, pioglitazone, englitazone, MCC-555, BRL49653 and the like); (1) preparations of interferon beta (interferon beta-1a, interferon beta-1b); (m) anticholinergic agents such as muscarinic antagonists (ipratropium and tiatropium); (n) current treatments for multiple sclerosis, including prednisolone, glatiramer, deoxyadenosine, mitoxantrone, methotrexate, and cyclophosphamide; (o) p38 kinase inhibitors; (p) other compounds such as 5-aminosalicylic acid and prodrugs thereof, antimetabolites such as azathioprine and 6-mercaptopurine, and cytotoxic cancer chemotherapeutic agents.

The weight ratio of the compound of the Formula I to the second active ingredient may be varied and will depend upon the effective dose of each ingredient. Generally, an effective dose of each will be used. Thus, for example, when a compound of the Formula I is combined with an NSAID the weight ratio of the compound of the Formula I to the NSAID will generally range from about 1000:1 to about 1:1000, preferably about 200:1 to about 1:200. Combinations of a compound of the Formula I and other active ingredients will generally also be within the aforementioned range, but in each case, an effective dose of each active ingredient should be used.

### Prodrugs

Some of the compounds of the invention are prodrugs which covert in vivo to the active moiety. For example, when R¹ is ethyl, the compounds of the invention covert in vivo to the corresponding acid. Such prodrugs are readily identifiable by one having ordinary skill in the art.

### Methods of Synthesis

Abbreviations that may be used in the following Schemes and Examples include: 4-DMAP: 4-dimethylaminopyridine; AcCN: acetonitrile; BOC: tert-butoxycarbonyl; BOC—ON:2-(tert-butoxycarbonyloxyimino)-2-phenylacetonitrile; BOP: benzotriazol-1-yloxy-tris(dimethylamino)-phosphonium hexafluorophosphate; brine: saturated NaCl solution; DIPEA: N,N-diisopropylethylamine; DMF: dimethylformamide; DMSO: dimethylsulfoxide; Et: ethyl; EtOAc: ethyl acetate; EtOH: ethanol; g or gm: gram; h or hr: hours; HATU: O-(7-azabenzotriazol-1-yl)-1,1,3,3-tetramethyluronium hexafluorophosphate; HBTU: O-(benzotriazol-1-yl)-1,1,3,3-tetramethyluronium hexafluorophosphate; HOAc: acetic acid; HOAt: 1-hydroxy-7-azabenzotriazole; HOBt: 1-hydroxybenzotriazole; HPLC: high pressure liquid chromatography; in vacuo: rotoevaporation; Me: methyl; MeOH: methanol; mg: milligram; MHz: megahertz; min: minutes; mL: milliliter; mmol: millimole; MS or ms: mass spectrum; MsCl: methanesulfonyl chloride; Ph: phenyl; Ph₃P: triphenylphosphine; PyBOP: (benzotriazol-1-yloxy)-tripyrrolidinophosphonium hexafluorophosphate; rt: room temperature; TEA: triethylamine; TFA: trifluoroacetic acid; THF: tetrahydrofuran.

The methysulfone derived compounds of the present invention may be prepared by the procedures illustrated in Scheme 1, and the phenylsulfone derived compounds may be prepared by the procedures illustrated in Example 7 and 8. In Scheme 1, a substituted pyridyl-4-carboxylic acid derivative A is treated with thionyl chloride to make the carboxylic acid chloride derivative which is subsequently reacted with a 4-amino-(L)-phenylalanine derivative to yield the amide B. The N—BOC-protecting group in B is removed with strong acid (TFA or HCl) to afford the free amine C. An appropriately substituted (L)-proline ester D is sulfonylated with 3-methylsulfonylbenzenesulfonyl chloride in the presence of base (DIPEA or Na₂CO₃) to yield sulfonamide E which, if containing an ester protecting group, is treated with hydroxide to afford the free acid. Amine C and acid E are reacted together in the presence of an appropriate coupling agent (eg., PyBOP, HBTU/HOAt, premade the acid chloride of E, etc.) to afford amide F. The ester in F can be hydrolyzed with hydroxide (if R¹ is n- or i-alkyl) or TFA or HCl (if R¹ is tert-butyl) to afford acid G.

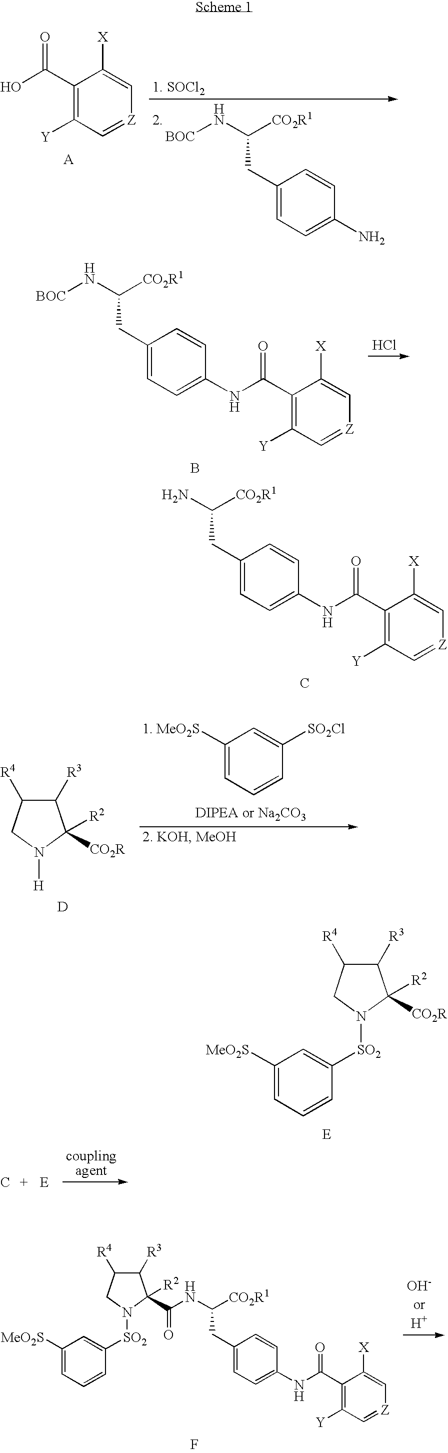

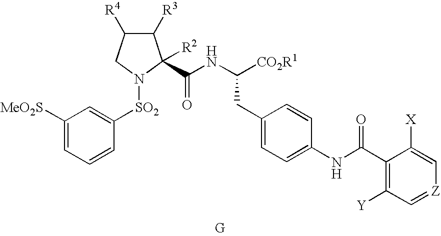

### Biological Evaluation

Compounds of formula I are potent antagonists of VLA-4 with significant and sustained receptor occupancy on VLA-4 bearing cells. The rate of dissociation of a test compound from VLA-4 on Jurkat cells may be determined by the method described in G. Doherty et al., Bioorganic & Medicinal Chemistry Letters, 13, 1891 (2003). Compounds of the present invention had half-lives of dissociation of greater than three hours (t_{1/2}>3 hr) in this assay, demonstrating they are tight binding inhibitors of VLA-4.

VLA-4 receptor occupancy after oral dosing in rats and dogs may be determined by the method described in D. R. Leone et al., J. Pharmacol. Exper. Therap., 305, 1150 (2003). Compounds of the present invention demonstrated sustained and significant receptor occupancy (>50%) after oral dosing.

Compounds of the present invention may be prepared by procedures detailed in the following examples. The examples provided are illustrative of the present invention and are not to be construed as limiting its scope in any manner:

### REFERENCE EXAMPLE 1

### 4-((3′,5′-Dichloroisonicotinoyl)amino)-(L)-phenylalanine, Ethyl Ester, Hydrochloride

### Step A: 4-Nitro-L-Phenylalanine, Ethyl Ester, Hydrochloride

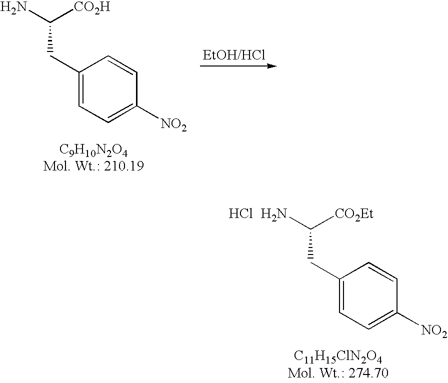

To 500 mL of absolute ethanol under nitrogen at 0° C. was added thionyl chloride (21 mL, 0.29 mol) over 5 min, and the clear solution was stirred at 0° C. for 10 min and then at room temperature for 30 min. 4-Nitro-L-phenylalanine (50.2 g, 0.24 mol) was added in one portion, and the mixture was refluxed overnight. The resulting mixture was concentrated in vacuo to give the title compound (60 g, 92%) as a white solid. ¹H NMR (400 MHz, CD₃OD) δ 8.21 (d, 2H), 7.54 (d, 2H), 4.39 (dd, 1H), 4.22 (q, 2H), 3.24-3.40 (m, 2H), 1.22 (t, 3H).

### Step B: N—BOC-4-Nitro-L-Phenylalanine, Ethyl Ester

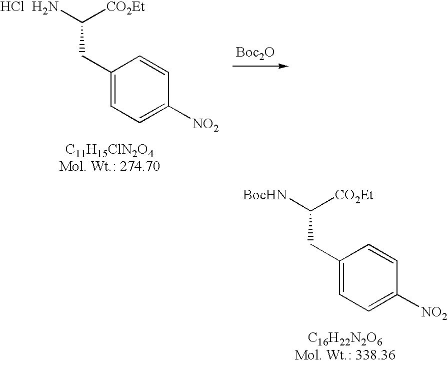

To a suspension of 4-nitro-L-phenylalanine ethyl ester hydrochloride (60 g, 0.22 mol) in methylene chloride (1.5 L) under nitrogen was added triethylamine (31 mL). After stirring at room temperature for 10 min, di-t-butyl dicarbonate (49 g, 0.22 mol) and 4-dimethylaminpyridine (0.1 g) was added, and the reaction was stirred at room temperature overnight. The reaction mixture was washed with 1 N HCl (2×200 mL), H₂O (2×200 mL) and brine (1×250 mL), dried over anhydrous Na₂SO₄, filtered and concentrated to afford the title compound (78 g, 100%). ¹H NMR (400 MHz, CDCl₃) δ 8.14 (d, 2H), 7.28 (d, 2H), 4.30-4.65 (m, 1H), 4.15 (q, 2H), 3.00-3.30 (m, 2H), 1.35 (s, 9H), 1.20 (t, 3H).

### Step C: N—BOC-4-amino-L-Phenylalanine, Ethyl Ester

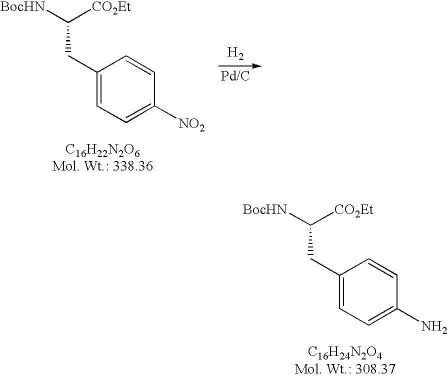

A solution of N—BOC-4-nitro-L-phenyl alanine ethyl ester (78.3 g, 0.22 mol) in absolute ethanol (300 mL) was purged with nitrogen, and 10% palladium on carbon (1.0 g) was added. After hydrogenated at 40-50 psi for 1 h, the reaction mixture was filtered through Celite, and the cake was washed with EtOH followed by EtOAc. The filtrate was concentrated, and the residue was purified by flash column chromatography on silica gel eluting with 4:1 to 1:1 EtOAc/Hexanes to afford the title compound (60 g 89% yield). ¹H NMR (400 MHz, CDCl₃) δ 6.90 (d, 2H), 6.63 (d, 2H), 4.20-4.50 (m, 1H), 4.14 (q, 2H), 3.76-3.00 (m, 2H), 1.36 (s, 9H), 1.20 (t, 3H).

### Step D: N—BOC-4-((3′,5′-dichloroisonicotinoyl)amino)-L-phenylalanine, Ethyl Ester

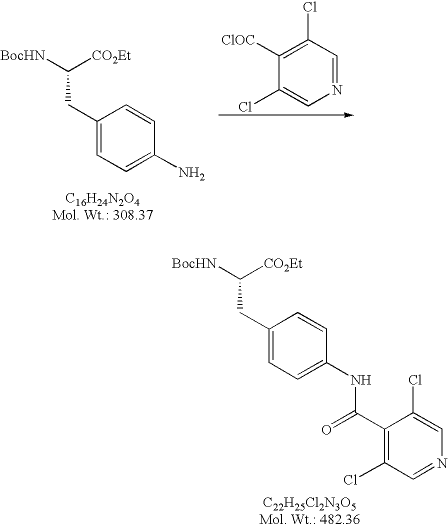

To a nitrogen flushed 500 mL round bottom flask was charged with 3,5-dichloro-isonicotinic acid (46.5 g, 0.24 mol), CH₂Cl₂ (150 mL), DMF (0.5 mL), and thionyl chloride (20 mL, 33.9 g 0.28 mol). After the slurry was refluxed for 5 h, additional thionyl chloride (5 mL, 0.70 mol) and CH₂Cl₂ (100 mL) were added, and the reaction was refluxed for additional 45 min. The reaction mixture was concentrated, and the residue was azeotroped with toluene to give the crude acyl chloride, which was used immediately. Thus, the crude acyl chloride was dissolved in CH₂Cl₂ (150 mL) and was added to N—BOC-4-amino-L-phenylalanine ethyl ester (60 g, 0.20 mol) and 4-methylmorpholine (44 mL, 0.40 mol) in CH₂Cl₂ (400 mL) at 0° C. over 5 min. After stirring at 0° C. for 1 h, the reaction was quenched with dilute aqueous NaHCO₃. The organic layer was separated and the aqueous layer was extracted with CH₂Cl₂ (500 mL). The organic layers were combined, dried over anhydrous MgSO₄ and concentrated in vacuo, and the residue was purified by flash column chromatography on silica gel eluting with 4:1 to 3:2 EtOAc/hexanes to afford the title compound (95 g, 100% yield). ¹H NMR (400 MHz, CD₃OD) δ 8.60 (s, 2H), 7.54 (d, 2H), 7.20 (d, 2H), 4.20-4.36 (m, 1H), 4.10 (q, 2H), 3.02-3.12 (m, 1H), 2.82-2.92 (m, 1H), 1.34/1.30 (s, 9H), 1.20 (t, 3H).

### Step E: 4-((3′,5′-Dichloroisonicotinoyl)amino)-(L)-phenylalanine, Ethyl Ester, Hydrochloride

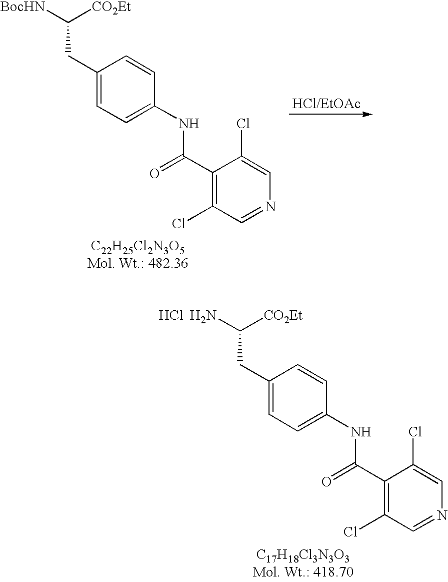

A solution of N—BOC-4-((3′,5′-dichloroisonicotinoyl)amino)-L-phenylalanine ethyl ester (95 g, 0.197 mmol) in EtOAc (1.2 L) was treated with a stream of hydrogen chloride gas over 2 h at room temperature. The resulting yellow suspension was diluted with hexanes (250 mL), cooled to 0° C. and filtered. The cake was washed with hexanes and dried in vacuo to afford the title compound as a yellow solid (80 g, 98%). ¹H NMR (400 MHz, CD₃OD) δ 8.64 (s, 2H), 7.66 (d, 2H), 7.30 (d, 2H), 4.28 (dd, 1H), 4.25 (q, 2H), 3.20 (q, 2H), 1.26 (t, 3H).

### REFERENCE EXAMPLE 2

### cis-Octahydroisoindole-1-carboxylic acid

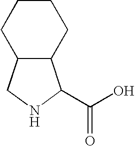

cis-N—BOC-octahydroisoindole-1-carboxylic acid (18.6 mmol) was dissolved in 4 M HCl in dioxane (46.5 mL) at rt. After stirring for 1 hr, the reaction was determined to be complete by TLC. The reaction mixture was concentrated in vacuo, triturated with Et₂O (2×70 mL) and then dried in vacuo to afford cis-octahydroisoindole-1-carboxylic acid (4.13 g) as a white solid.

500 MHz ¹HNMR (CD₃OD) δ 4.40 (d, J=5.5 Hz, 1H); 3.39 (m, 1H); 3.31 (m, 1H); 2.70 (m, 1H); 2.58; (m, 1H); 1.78-1.14 (m, 8H).

### EXAMPLE 1

### (R,S,S)—N—{N-[(3-Methylsulfonylbenzene)sulfonyl]-4(R)-cyclobutylamino-(L)-prolyl}-4-[(3′,5′-dichloro-isonicotinoyl)amino]-(L)-phenylalanine, Ethyl Ester

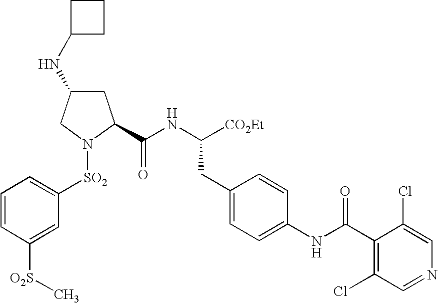

### Step A: N—BOC-4(R)-cyclobutylamino-L-proline, Methyl Ester

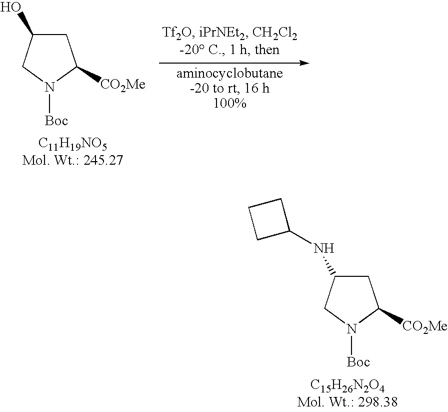

To a solution of N—BOC-cis-hydroxy-L-proline methyl ester (60 g, 0.25 mol) and N,N-diisopropylethylamine (100 mL, 0.57 mol) in methylene chloride (800 mL) at −20° C. was added trifluoromethanesulfonic anhydride over 45 min. After stirring at −20° C. for additional 45 min, cyclobutylamine (55 g, 0.78 mol) was added in one portion, and the reaction was allowed to warm up to room temperature overnight. The reaction was quenched with 1 N NaOH (250 mL) and saturated aqueous NaHCO₃ (250 mL). The organic layer was separated, washed with brine, dried with magnesium sulfate, filtered and concentrated, and the residue was purified by flash column chromatography on silica gel eluting with CH₂Cl₂ to EtOAc to 5% MeOH/EtOAc) to afford the title compound (77 g, 100%). ¹H NMR (400 MHz, CD₃OD) δ 4.8 (br s, 1H), 4.24-4.32 (m, 1H), 3.67/3.68 (s, 3H), 3.56-3.64 (m, 1H), 3.1-3.38 (m, 3H), 2.12-2.21 (m, 2H), 2.00-2.12 (m, 2H), 1.54-1.82 (m, 4H), 1.36/1.43 (s, 9H).

### Step B: N—[N—BOC-4(R)-cyclobutylamino-(L)-prolyl]-4-[(3′,5′-dichloro-isonicotinoyl)amino]-(L)-phenylalanine, Ethyl Ester

To a solution of N—BOC-4(R)-cyclobutylamino-(L)-proline methyl ester (77 g, 0.25 mol) in CH₃CN (350 mL) and water (150 mL) was added lithium hydroxide monohydrate (21 g, 0.50 mol), and the suspension was stirred at room temperature overnight. The reaction mixture was diluted to a total volume of 1 L with CH₃CN, cooled to 0° C. and filtered, and the filtrate was obtained as a solution of the lithium salt of N—BOC-4(R)-cyclobutylamino-(L)-proline. A portion of the above filtrate (484 mL, 0.22 M, 0.11 mol) was concentrated, and to the residue was added water (1 L), CH₂Cl₂ (1 L), EDC (20.8 g, 0.11 mol), 1-hydroxybenzotriazole (14.6 g, 0.11 mol) and a solution of 4-((3′,5′-dichloro-isonicotinoyl)amino)-(L)-phenylalanine ethyl ester hydrochloride (39 g, 0.090 mol) in water (1 L). The biphasic mixture was stirred vigorously at room temperature for 4 h. The organic layer was separated and the aqueous layer was extracted with CH₂Cl₂ (0.5 L). The combined organic layers were dried over Na₂SO₄, filtered and concentrated, and the residue was purified by flash column chromatography on silica gel eluting with EtOAc to EtOAc/MeOH/NH₄OH (98:1:1) to afford the title compound (48 g 78%). ¹H NMR (400 MHz, CD₃OD) δ 8.64 (s, 2H), 7.57 (d, 2H), 7.20-7.30 (m, 2H), 4.80 (br s, 1H), 4.58-4.70 (m, 1H), 4.08-4.30 (m, 3H), 3.56-3.64 (m, 1H), 2.90-3.30 (m, 5H), 3.00-3.08 (m, 1H), 1.5-2.2 (m, 8H), 1.22 (br s, 9H), 1.2 (t, 3H).

### Step C: N-[4(R)-Cyclobutylamino-(L)-prolyl]-4-[(3′,5′-dichloro-isonicotinoyl)amino]-(L)-phenylalanine, Ethyl Ester, Hydrochloride

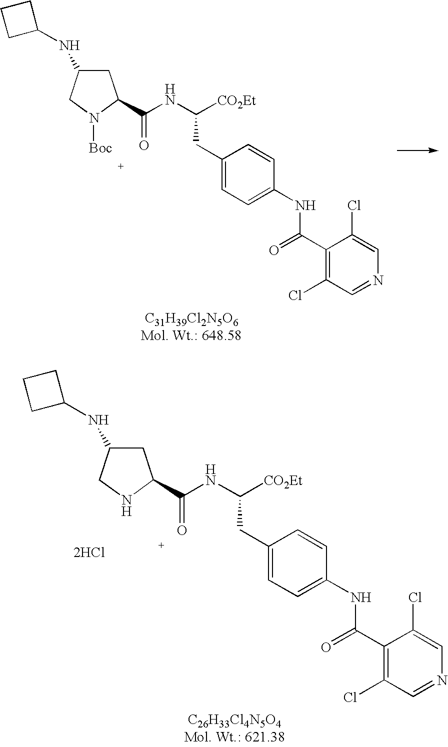

To a solution of N—[N—BOC-4(R)-cyclobutylamino-(L)-prolyl]-4-[(3′,5′-dichloro-isonicotinoyl)amino]-(L)-phenylalanine ethyl ester in ethanol (300 mL) was added 4 M HCl in dioxane (250 mL). After stirring at room temperature overnight, the clear yellow solution was concentrated, and the residue was triturated with ether (1 L). The resulting suspension was stirred for at room temperature for 2 h, and the precipitate was collected by filtration. The cake was washed with ether and dried to afford the title compound (45.6 g, 100% yield). ¹H NMR (400 MHz, CD₃OD) δ 8.86 (d, 1H), 8.62 (s, 2H), 7.58 (d, 2H), 7.26 (d, 2H), 4.80 (br s, 1H), 4.70 (m, 1H), 4.64 (dd, 1H), 4.15 (q, 2H), 3.80-3.95 (m, 3H), 3.5 (dd, 1H), 3.24 (dd, 1H), 3.00 (dd, 1H), 2.50-2.70 (m, 2H), 2.20-2.40 (m, 4H), 1.8-2.0 (m, 2H), 1.22 (t, 3H).

### Step D: N—{N-[(3-Methylsulfonylbenzene)sulfonyl]-4(R)-cyclobutylamino-(L)-prolyl}-4-[(3′,5′-dichloro-isonicotinoyl)amino]-(L)-phenylalanine Ethyl Ester

To a suspension of N-[4(R)-cyclobutylamino-(L)-prolyl]-4-[(3′,5′-dichloro-isonicotinoyl)amino]-(L)-phenylalanine ethyl ester hydrochloride (18 g, 30 mmol), 3-methylsulfonylbenzenesulfonyl chloride (7.64 g, 30 mmol), 4-dimethylaminopyridine (50 mg) in tetrahydrofuran (80 mL) and methylene chloride (80 mL) at 0° C. was added diisopropylethylamine (21 mL, 0.12 mol). The reaction was allowed to warm up to room temperature over 3 h, and the resulting mixture was concentrated. The residue was dissolved in ethyl acetate (400 mL), washed with 1 N sodium hydroxide and brine and concentrated to dryness. The residue was purified by flash column chromatography on silica gel with 2 N ammonia in methanol/EtOAc (0 to 3%) to give the title compound (21.4 g, 93%).

¹H NMR (500 MHz, CD₃OD): δ 8.63 (s, 2H), 8.31 (s, 1H), 8.23 (m, 2H), 7.95 (d, 1H), 7.80 (t, 1H), 7.62 (d, 2H), 7.32 (d, 2H), 4.61 (dd, 1H), 4.47 (dd, 1H), 4.14 (m, 2H), 3.68 (dd, 1H), 3.57 (m, 1H), 3.42 (dd, 1H), 3.20 (s, 3H), 3.21 (m, 1H), 3.05 (dd, 1H), 2.19 (m, 2H), 1.94 (m, 1H), 1.87 (m, 2H), 1.73 (m, 2H), 1.22 (t, 3H). LC-MS: calculated for C33H37Cl2N5O8S2 765, observed m/e 766 (M+H)⁺ (2.85 min).

### EXAMPLE 2

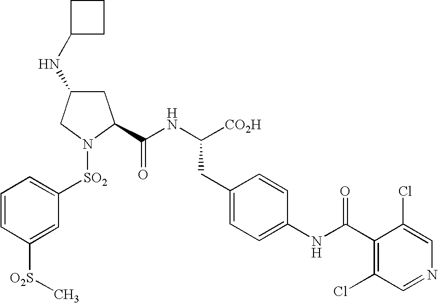

### (R,S,S)—N—{N-[(3-Methylsulfonylbenzene)sulfonyl]-4(R)-cyclobutylamino-(L)-prolyl}-4-[(3′,5′-dichloro-isonicotinoyl)amino]-(L)-phenylalanine

To a solution of N—{N-[(3-methylsulfonylbenzene)sulfonyl]-4(R)-cyclobutylamino-(L)-prolyl}-4-[(3′,5′-dichloro-isonicotinoyl)amino]-(L)-phenylalanine ethyl ester (compound of Example 1, 4.794 g, 6.378 mmol) in 40 mL of acetonitrile was added 15.93 mL of 1N NaOH. After stirring at room temperature for 2 h, the reaction was quenched with 8 mL of 2N HCl to make the reaction solution slightly acidic. The reaction was diluted with 450 mL of THF/EtOAc (1:3 v/v) and washed with water (100 mL×2). After evaporation of the solvent, the residue was dissolved in 100 mL of acetonitrile and lyophilized to give 4.6 g (99%) of the title product. ¹H NMR (500 MHz, CD₃OD): δ 8.62 (s, 2H), 8.33 (s, 1H), 8.20 (d, 1H), 7.90 (d, 1H), 7.76 (t, 1H), 7.60 (d, 2H), 7.34 (d, 2H), 4.49 (dd, 1H), 4.44 (dd, 1H), 3.70 (dd, 1H), 3.63 (m, 1H), 3.52 (m, 1H), 3.22 (m, 2H), 3.20 (s, 3H), 3.01 (dd, 1H), 2.27 (m, 2H), 1.97 (m, 2H), 1.80 (m, 1H). LC-MS: calculated for C31H33Cl2N5O8S2 737, observed m/e 738 (M+H)⁺ (2.54 min).

### EXAMPLE 3

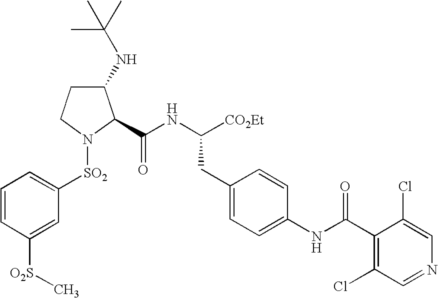

### N—{N-[(3-Methylsulfonylbenzene)sulfonyl]-3(S)-tert-butylamino-(L)-prolyl}-4-[(3′,5′-dichloro-isonicotinoyl)amino]-(L)-phenylalanine, Ethyl Ester

### Step A: N-[(3-Methylsulfonylbenzene)sulfonyl]-3(S)-hydroxy-(L)-proline, Methyl Ester

To a solution of (3S)-hydroxy-(L)-proline (Acros, 1.223 g, 9.324 mmol) and sodium carbonate (2.08 g, 19.63 mmol) in 30 mL of water at 0° C. was added powdered 3-methylsulfonylbenzenesulfonyl chloride (2.5 g, 9.815 mmol). After stirring at room temperature overnight, the reaction mixture was acidified with concentrated hydrochloric acid (pH=3), and the product was extracted with ethyl acetate (3×30 mL). The organic extracts were dried (MgSO₄), filtered and concentrated to dryness. The residue was then dissolved in methylene chloride (10 mL) and methanol (10 mL), and was added trimethylsilyldiazomethane (2 M in ether) at 0° C. until a yellow color persisted. After stirring at room temperature for 15 min, the mixture was concentrated to dryness to give the title compound (2.6 g, 77%).

LC-MS: calculated for C13H17NO7S2 363, observed m/e 364 (M+H)⁺ (2.1 min).

### Step B: N-[(3-Methylsulfonylenzene)sulfonyl]-3(S)-methanesulfonyloxy-(L)-proline, Methyl Ester

To a solution of N-[(3-methylsulfonylbenzene)sulfonyl]-3(S)-hydroxy-(L)-proline, methyl ester (2.6 g, 7.16 mmol) in 24 mL of dichloromethane at 0° C. was added triethylamine (1.2 mL, 8.59 mmol) and methanesulfonyl chloride (0.61 mL, 7.87 mmol). After stirring at 0° C. for 20 min, the reaction was quenched with 30 mL of aqueous sodium bicarbonate. After stirring for 15 min, the reaction mixture was partitioned between ethyl acetate (100 mL) and aqueous sodium bicarbonate (50 mL). The organic layer was separated, washed with brine and concentrated to dryness to give the product (3.156 g, 99%).

LC-MS: calculated for C14H19NO9S3 441, observed m/e 442 (M+H)⁺ (2.5 min).

### Step C: N-[(3-Methylsulfonylbenzene)sulfonyl]-2,3-dehydroproline, Methyl Ester

To a solution of N-[(3-Methylsulfonylbenzene)sulfonyl]-3(s)-methanesulfonyloxy-(L)-proline, methyl ester (3.156 g, 7.15 mmol) in 24 mL of acetonitrile was added triethylamine (3.985 mL, 28.6 mmol). After heating at 75° C. for 4 h, the reaction mixture was cooled to room temperature and was concentrated. The residue was dissolved in ethyl acetate (100 mL) and was washed with 1 N aqueous sodium hydroxide and brine, and concentrated to dryness to give the title compound (2.4 g, 97%). LC-MS: calculated for C13H15NO6S2 345, observed m/e 346 (M+H)⁺(2.53 min).

### Step D: N-[(3-Methylsulfonylbenzene)sulfonyl]-3-tert-butylaminoproline, Methyl Ester

To a suspension of N-[(3-methylsulfonylbenzene)sulfonyl]-2,3-dehydroproline, methyl ester (2.4 g, 6.95 mmol) in 18 mL of cyclohexane and 6 mL of tert-butanol was added tert-butylamine (7.3 mL, 69.5 mmol). After heating at 50° C. for 48 h, the reaction mixture was cooled to room temperature and was concentrated. The solid residue was triturated with hexane and was collected by filtration to give the title compound (1.63 g, 56%). ¹H NMR (400 MHz, CD₃OD): δ 8.38 (s, 1H), 8.22 (d, 1H), 8.20 (d, 1H), 7.87 (dd, 1H), 3.99 (d, 1H), 3.75 (s, 3H), 3.50 (m, 3H), 3.20 (s, 3H), 2.17 (m, 1H), 1.62 (m, 1H), 0.97 (s, 9H). LC-MS: calculated for C17H26N2O6S2 418, observed m/e 419 (M+H)⁺ (2.0 min).

### Step E: N—{N-[(3-methylsulfonylbenzene)sulfonyl]-3-tert-butylaminoprolyl}-4-[(3′,5′-dichloro-isonicotinoyl)amino]-(L)-phenylalanine, Ethyl Ester

To a solution of N-[(3-methylsulfonylbenzene)sulfonyl]-3-tert-butylaminoproline methyl ester (Step D, 1.63 g, 3.883 mmol) in 10 mL of acetonitrile/water (2.5:1) was added lithium hydroxide monohydrate (407 mg, 9.71 mmol). After stirring at room temperature for 2.5 h, the reaction was quenched by addition of 2 N aqueous hydrochloric acid (5 mL, 10 mmol), and the reaction mixture was lyophilized to give the crude lithium salt, which was used without further purification. Thus, to a suspension of the crude lithium salt in 13 mL of dry dimethylformamide was added 4-[(3′,5′-dichloroisonicotinoyl)amino]-(L)-phenylalanine ethyl ester hydrochloride salt (1.79 g, 4.27 mmol), (benzotriazol-1-yloxy)tripyrrolidinophosphonium hexafluorophosphate (3.03 g, 5.83 mmol) and N-methylmorpholine (1.7 mL, 15.5 mmol). After stirring at room temperature for 2.5 h, the reaction mixture was diluted with ethyl acetate (50 mL), washed with water (30 mL×2) and brine, dried over anhydrous magnesium sulfate, filtered and concentrated to dryness. The residue was purified by flash chromatography on silica gel to give the title compound. LC-MS: calculated for C33H39Cl2N5O8S2 767, observed m/e 768 (M+H)⁺ (2.8 min).

### EXAMPLE 4

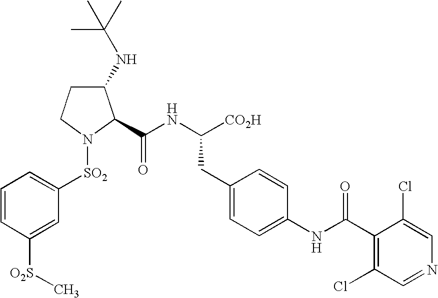

### N—{N-[(3-Methylsulfonylbenzene)sulfonyl]-3-tert-butylaminoprolyl}-4-[(3′,5′-dichloro-isonicotinoyl)amino]-(L)-phenylalanine

To a solution of N—{N-[(3-methylsulfonylbenzene)sulfonyl]-3-tert-butylaminoprolyl}-4-[(3′,5′-dichloro-isonicotinoyl)amino]-(L)-phenylalanine ethylester (the title compound of Example 3, 50 mg, 0.065 mmol) in 1 mL of acetonitrile was added sodium hydroxide (1 N, 0.164 mL, 0.164 mmol), and the reaction was stirred at room temperature for 60 min. The reaction was quenched with 0.2 ml of formic acid and diluted with 0.5 mL of DMSO and 0.3 mL of water. This reaction mixture was subjected to reverse HPLC and the fractions containing the product were collected and lyophilized to give the title compound. LC-MS: calculated for C31H35Cl2N5O8S2 739, observed m/e 740 (M+H)⁺ (2.5 min).

### EXAMPLE 5

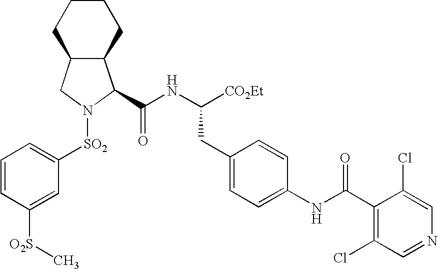

### (S,R,S,S)—N—{N-[(3-Methylsulfonylbenzene)sulfonyl]octahydroisoindol-1-carboxyl}-4-[(3′,5′-dichloro-isonicotinoyl)amino]-(L)-phenylalanine, Ethyl Ester

### Step A: (S,R,S,S)—N—(N—Boc-octahydroisoindol-1-carboxyl)-4-[(3′,5′-dichloro-isonicotinoyl)amino]-(L)-phenylalanine, Ethyl Ester

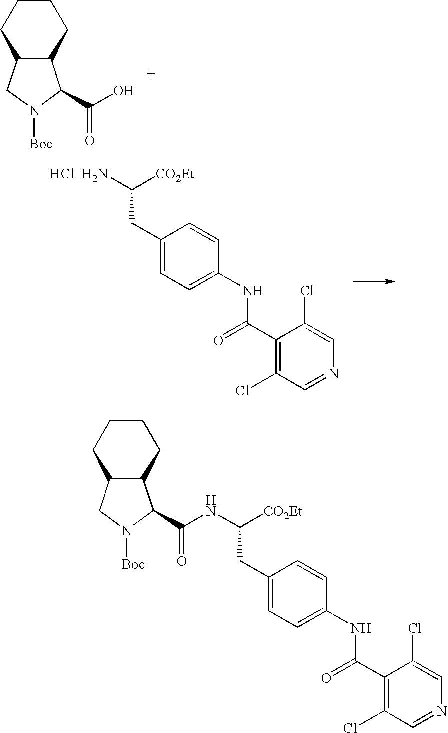

To a slurry of racemic N-Boc-octahydroisoindol-1-carboxylic acid (from Neosystem, 6 g, 22.28 mmol, 1 equiv), the amine HCl salt from Step E of Reference Example 1 (9.3 g, 22.28 mmol, 1 equiv), and PYBOP (17.4 g, 33.42 mmol, 1.5 equiv) in 100 mL of CH₂Cl₂ was added N-methyl morphorline (8.52 mL, 78 mmol, 3.5 equiv) at 0° C. The cooling bath was removed, and the reaction solution, which became clear now, was stirred overnight at rt. LCMS showed the reaction went to completion. Then the reaction solution was loaded to a 65M silica column and eluted with 2-10% THF in dichloromethane. Collect the higher Rf isomer (the desired diastereomer) and discard the lower Rf isomer. The diastereomer mixture fractions in the middle were collected separately and columned again using the above conditions. The two batches of the top Rf isomer were combined to give 5.64 g (yield 80%) of compound 3. The two diastereomers can also be separated nicely by ChiralPak AD 4.6×250 mm 10 u (40% isopropanol/60% heptane, isocratic. Retention time: undesired isomer 8.1 min, desired isomer 10.5 min). ¹H NMR (500 MHz, CD₃OD): δ 8.65 (s, 2H); 7.60 (d, J=8.5 Hz, 2H); 7.31 (d, J=8.5 Hz, 2H); 4.70 (broad s, 1H); 4.21 (d, J=6 Hz 1H); 4.15 (m, 2H); 3.50 (m, 1H); 3.15 (m, 1H); 2.96 (m, 1H); 2.37 (m, 1H); 2.27 (m, 1H); 1.71-1.08 (m, 13H); 1.27 (s, 9H). LCMS m/e 632 (M⁺)/532 (M⁺-Boc), 3.49 min.

### Step B: (S,R,S,S)—N-(octahydroisoindol-1-carboxyl)-4-[(3′,5′-dichloro-isonicotinoyl)amino]-(L)-phenylalanine Ethyl Ester HCl Salt

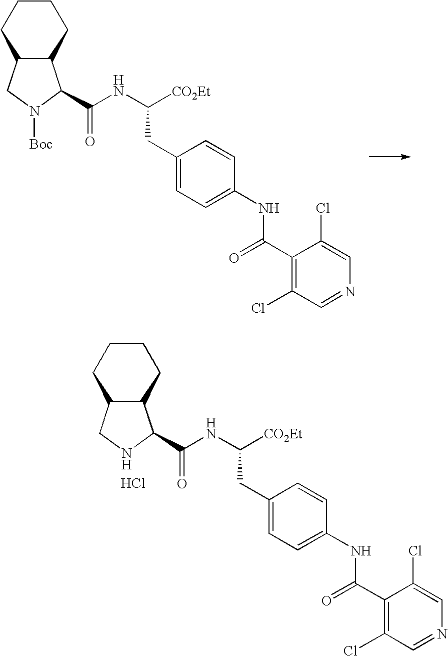

To the product of Step A (5.64 g, 8.9 mmol) in 50 mL of EtOAc/CH₂Cl₂ (1:1 v/v) was added 20 mL of 4N HCl in 1,4-dioxane. The reaction mixture was stirred overnight and concentrated in vacuum to afford the title compound (5.02 g, yield 99%). ¹H NMR (500 MHz, CD₃OD): δ 8.65 (s, 2H); 7.59 (d, J=8.5 Hz, 2H); 7.27 (d, J=8.5 Hz, 2H); 4.80 (m, 1H); 4.18 (q, J=7 Hz, 2H); 3.28 (m, 2H); 2.98 (m, 1H); 2.60 (m, 2H); 1.78-1.55 (m, 5H); 1.28 (m, 2H); 1.25 (t, J=7 Hz, 3H); 1.03 (m, 1H). LCMS in/e 532 (M⁺), 2.63 min.

### Step C: (S,R,S,S)—N—{N-[(3-Methylsulfonylbenzene)sulfonyl]octahydroisoindol-1-carboxyl}-4-[(3′,5′-dichloro-isonicotinoyl)amino]-(L)-phenylalanine, Ethyl Ester

To a suspension of (S,R,S,S)—N-(octahydroisoindol-1-carboxyl)-4-[(3′,5′-dichloro-isonicotinoyl)amino]-(L)-phenylalanine ethyl ester HCl salt (product of Step B, 1.522 g, 2.853 mmol) and 3-methylsulfonylbenzenesulfonyl chloride (0.8 g, 3.138 mmol) in tetrahydrofuran (5 mL) and methylene chloride (5 mL) at 0° C. was added triethylamine (1.19 mL, 8.56 mmol). The reaction was allowed to warm up to room temperature over 2 h, and the resulting mixture was concentrated. The residue was dissolved in ethyl acetate (50 mL), washed with 1 N sodium hydroxide and brine and concentrated to dryness. The residue was purified by flash column chromatography on silica gel with EtOAc/hexanes (50 to 100%) to give the title compound (1.687 g, 79%).

¹H NMR (500 MHz, CD₃OD): δ 8.62 (s, 2H), 8.38 (s, 1H), 8.24 (d, 1H), 8.14 (d, 1H), 7.87 (dd, 1H), 7.63 (d, 2H), 7.32 (d, 2H), 4.80 (dd, 1H), 4.18 (q, 2H), 3.51 (dd, 1H), 3.40 (m, 1H), 3.25 (dd, 1H), 3.20 (s, 3H), 3.05 (dd, 2H), 2.25 (m, 1H), 1.73 (m, 1H), 1.58-1.35 (m, 5H), 1.28 (t, 3H), 1.06-0.97 (m, 4H). LC-MS: calculated for C33H36Cl2N4O8S2 750, observed m/e 751 (M+H)⁺ (3.4 min).

### EXAMPLE 6

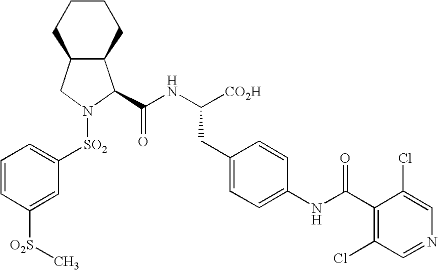

### (S,R,S,S)—N—{N-[(3-Methylsulfonylbenzene)sulfonyl]octahydroisoindol-1-carboxyl}-4-[(3′,5′-dichloro-isonicotinoyl)amino]-(L)-phenylalanine

The title compound was prepared following procedures described for Example 2 replacing the starting material with the compound from Example 5. ¹H NMR (500 MHz, CD₃OD): δ 8.62 (s, 2H), 8.39 (s, 1H), 8.23 (d, 1H), 8.14 (d, 2H), 7.83 (dd, 1H), 7.62 (d, 2H), 7.34 (d, 2H), 4.78 (dd, 1H), 4.18 (d, 1H), 3.49 (dd, 1H), 3.39 (m, 1H), 3.20 (s, 3H), 3.06 (m, 1H), 2.28 (m, 1H), 1.76 (m, 1H), 1.60-135 (m, 4H), 1.26-1.00 (m, 4H). LC-MS: calculated for C31H32Cl2N4O8S2 722, observed m/e 723 (M+H)⁺ (3.0 min).

### EXAMPLE 7

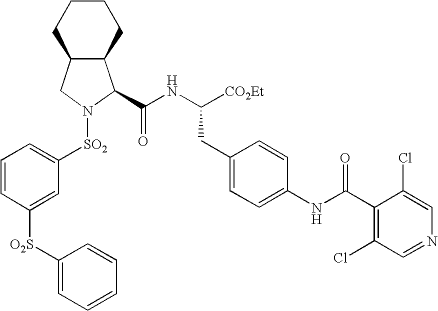

### (S,R,S,S)—N—{N-[(3-Phenylsulfonylbenzene)sulfonyl]octahydroisoindol-1-carboxyl}-4-[(3′,5′-dichloro-isonicotinoyl)amino]-(L)-phenylalanine, Ethyl Ester

### Step A: (S,R,S,S)—N—{N-[(3-Iodolbenzene)sulfonyl]octahydroisoindol-1-carboxyl}-4-[(3′,5′-dichloro-isonicotinoyl)amino]-(L)-phenylalanine, Ethyl Ester

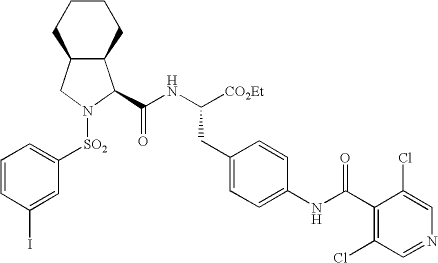

The title compound was prepared following procedures described for Example 5, Step C replacing 3-methylsulfonylbenzenesulfonyl chloride with 3-iodobenzenesulfonyl chloride. ¹H NMR (500 MHz, CD₃OD): δ 8.62 (s, 2H), 8.18 (s, 1H), 8.02 (d, 1H), 7.85 (d, 1H), 7.62 (d, 2H), 7.38 (dd, 1H), 7.28 (d, 2H), 4.80 (m, 1H), 4.20 (q, 2H), 3.40 (m, 2H), 3.25 (m, 2H), 3.05 (dd, 1H), 2.21 (m, 1H), 1.63 (m, 1H), 1.50 (m, 2H), 1.33 (m, 2H), 1.25 (t, 3H), 1.18 (m, 1H), 0.94 (m, 2H). LC-MS: calculated for C32H33Cl21N4O6S 798, observed m/e 799 (M+H)⁺ (3.8 min).

### Step B: (S,R,S,S)—N—{N-[(3-Phenylsulfonylbenzene)sulfonyl]octahydroisoindol-1-carboxyl}-4-[3′,5′-dichloro-isonicotinoyl)amino]-(L)-phenylalanine, Ethyl Ester

The title compound was prepared following procedures described in Org. Lett. 2002, 4, pp 4423-4425. Thus, (S,R,S,S)—N—{N-[(3-Iodolbenzene)sulfonyl]octahydroisoindol-1-carboxyl}-4-[(3′,5′-dichloroisonicotinoyl)amino]-(L)-phenylalanine, ethyl ester (from Step A, 53 mg, 0.066 mmol), phenylsulfinic acid sodium salt (32.7 mg, 0.199 mmol), (CuOTf)₂ benzene complex (10 mg, 0.0199 mmol), and N,N-dimethyl ethyldiamine (0.005 mL, 0.04 mmol) were dissolved in 0.5 mL of DMSO (dried over 4A MS). The reaction mixture was heated at 110° C. overnight. Then the reaction mixture was diluted with EtOAc (10 mL), and washed with water (10 mL×2) and brine. After dried over MgSO₄, the solvent was evaporated, and the residue was purified on SiO₂ with EtOAc/hexanes (60%-100%) to give the title compound (18 mg, 34%). ¹HNMR (500 MHz, CD₃OD): δ 8.61 (s, 2H), 8.38 (s, 1H), 8.24 (d, 1H), 8.01 (d, 2H), 7.81 (dd, 1H), 7.67 (d, 1H), 7.61 (m, 4H), 7.31 (d, 2H), 4.80 (m, 1H), 4.18 (q, 2H), 3.36 (m, 2H), 3.24 (dd, 2H), 3.06 (dd, 1H), 2.16 (m, 1H), 1.47 (m, 3H), 1.33 (m, 2H), 1.27 (t, 3H), 1.19 (m, 2H), 1.02-0.91 (m, 3H). LC-MS: calculated for C38H38Cl2N4O8S2 812, observed m/e 813 (M+H)⁺ (3.7 min).

### EXAMPLE 8

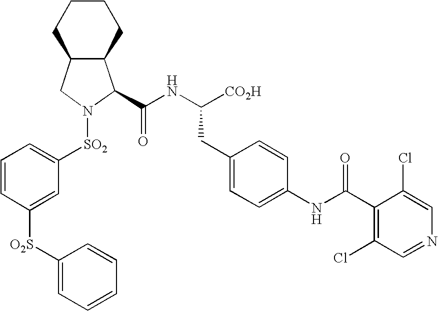

### (S,R,S,S)—N—{N-[(3-Phenylsulfonylbenzene)sulfonyl]octahydroisoindol-1-carboxyl}-4-[(3′,5′-dichloro-isonicotinoyl)amino]-(L)-phenylalanine

The title compound was prepared following procedures described for Example 2 replacing the starting material with the compound from Example 7. ¹H NMR (500 MHz, CD₃OD): δ 8.62 (s, 2H), 8.38 (s, 1H), 8.23 (d, 1H), 8.03 (d, 2H), 7.80 (dd, 1H), 7.68 (m, 1H), 7.60 (m, 4H), 7.34 (d, 2H), 4.78 (dd, 1H), 4.08 (d, 1H), 3.37 (m, 1H), 3.05 (m, 2H), 2.18 (m, 1H), 1.52 (dm, 3H), 1.38-0.95 (m, 6H). LC-MS: calculated for C36H34Cl2N4O8S2 784, observed m/e 785 (M+H)⁺ (3.5 min).

### EXAMPLE 9

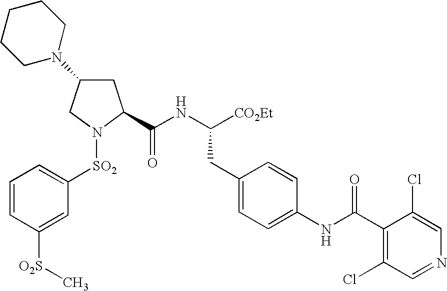

### N—{N-[(3-Methylsulfonylbenzene)sulfonyl]-4(R)-piperidino-(L)-prolyl}-4-[(3′,5′-dichloro-isonicotinoyl)amino]-(L)-phenylalanine, Ethyl Ester

### Step A: N-(tert-butoxycarbonyl)-4(R)-piperidino-L-proline, Methyl Ester

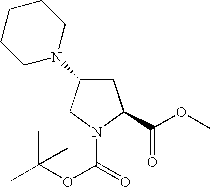

To a solution of N-(tert-butoxycarbonyl)-cis-hydroxy-L-proline methyl ester (1.5 g, 6.1 mmol) in CH₂Cl₂ (50 mL) was added N,N-diisopropylethylamine (2.7 mL, 15.25 mol). The reaction mixture was cooled to −78° C. and trifluoromethanesulfonic anhydride (1.4 mL, 8.5 mmol) was added dropwise to the solution. After stirring for 1 h, the mixture was warmed to −20° C. and piperidine (1.8 mL, 18.3 mmol) was added dropwise. The solution was warmed to room temperature and stirred overnight. Water was added to the solution and the aqueous layer was extracted with CH₂Cl₂. The combined organic layers were dried over MgSO₄ and concentrated. The residue was purified by flash chromatography to give N-(tert-butoxycarbonyl)-4(R)-piperidino-L-proline methyl ester as an oil. ¹H-NMR (CDCl₃, 500 MHz) δ 4.44-4.31 (m, 1H), 3.87-3.75 (m, 1H), 3.72 (s, 3H), 3.45-2.90 (m, 3H), 2.51-2.15 (m, 5H), 1.71-1.55 (m, 4H), 1.48-1.39 (m, 1H). MS (ESI) 313.3 (MH⁺).

### Step B: N-((3-Methylsulfonylbenzene)sulfonyl)-4(R)-piperidino-L-proline, Methyl Ester

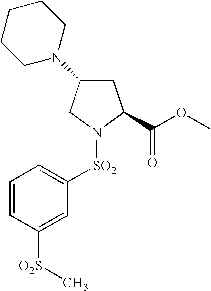

N-(tert-butoxycarbonyl)-4(R)-piperidino-L-proline methyl ester (388 mg, 1.24 mmol) was dissolved in CH₂Cl₂ (7.5 mL) and trifluoroacetic acid (2.5 mL) was added. After stirring for 3 h at room temperature, the solution was concentrated and further dried under high vacuum. The crude material was dissolved in CH₂Cl₂ (10 mL) and triethylamine (0.5 mL, 3.6 mmol) followed by 3-methylsulfonylbenzenesulfonyl chloride (500 mg, 1.96 mmol) were added. After stirring for 2 h at room temperature, the reaction was concentrated and purified by silica gel chromatography to give N-((3-methylsulfonylbenzene)sulfonyl)-4(R)-piperidino-L-proline methyl ester, which was used directly in the next step. MS (ESI) 431.0 (MH⁺).

### Step C: N—{N-[(3-Methylsulfonylbenzene)sulfonyl]-4(R)-piperidino-(L)-prolyl}-4-[(3′,5′-dichloro-isonicotinoyl)amino]-(L)-phenylalanine, Ethyl Ester

To a solution of N-((3-methylsulfonylbenzene)sulfonyl)-4(R)-piperidino-L-proline methyl ester (320 mg, 0.74 mmol) in acetonitrile/water (6 mL, 2/1) was added lithium hydroxide monohydrate (100 mg, 2.4 mmol). After stirring at room temperature for 30 min, the reaction was neutralized with 1N HCl (2.4 mL, 2.4 mmol), concentrated to dryness and used directly in the next step. MS (ESI) 417.1 (MH⁺).

To a suspension of the above acid in DMF (5 mL) was added N-methyl morpholine (0.25 mL, 2.3 mmol), HATU (310 mg, 0.82 mmol), and 4-[(3′,5′-dichloroisonicotinoyl)amino]-(L)-phenylalanine ethyl ester hydrochloride (310 mg, 0.74 mmol). After stirring at room temperature for 2 h, the reaction was slowly added to water (50 mL) and the resulting precipitate was filtered and dried. Half of this material was taken directly to the next step, while the remainder was purified by preparative reversed-phase HPLC. The product fractions were lyophilized to give the title compound as a white powder. ¹H-NMR (DMSO-d₆, 500 MHz) δ 10.84 (s, 1H), 8.78 (s, 2H), 8.41 (d, 1H), 8.26-8.25 (m, 1H), 8.24 (s, 1H), 8.13-8.11 (m, 1H), 7.91-7.88 (m, 1H), 7.58 (d, 2H), 7.27 (d, 2H), 4.51-4.49 (m, 1H), 4.36-4.34 (m, 1H), 4.11-4.07 (m, 2H), 3.60-3.57 (m, 1H), 3.34 (s, 3H), 3.05-3.03 (m, 2H), 2.92-2.89 (m, 1H), 2.78-2.81 (m, 1H), 2.21-2.11 (m, 4H), 1.82-1.79 (m, 1H), 1.63-1.60 (m, 1H), 1.35-1.27 (m, 6H), 1.17 (t, 3H). MS (ESI) 780.2 (M⁺).

### EXAMPLE 10

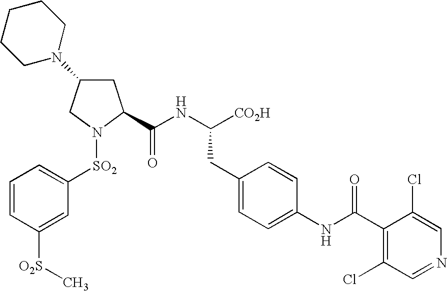

### N—{N-[(3-Methylsulfonylbenzene)sulfonyl]-4(R)-piperidino-(L)-prolyl}-4-[(3′,5′-dichloro-isonicotinoyl)amino]-(L)-phenylalanine

To a solution of N—{N-[(3-methylsulfonylbenzene)sulfonyl]-4(R)-piperidino-(L)-prolyl}-4-[(3′,5′-dichloro-isonicotinoyl)amino]-(L)-phenylalanine ethyl ester (Compound of Example 9, 133 mg, 0.17 mmol) in acetonitrile/water (3 mL, 2/1) was added lithium hydroxide monohydrate (15 mg, 0.35 mmol). After stirring at room temperature for 30 min, the reaction was quenched with 5 drops of formic acid. The reaction was directly purified by preparative reversed-phase HPLC and the product fractions were lyophilized to provide the title compound as a white powder. ¹H-NMR (DMSO-d₆, 500 MHz) δ 12.86 (br s, 1H), 10.84 (s, 1H), 8.78 (s, 2H), 8.27 (s, 1H), 8.26-8.22 (m, 2H), 8.14-8.12 (m, 1H), 7.90-7.87 (m, 1H), 7.58 (d, 2H), 7.27 (d, 2H), 4.47-4.45 (m, 1H), 4.38-4.36 (m, 1H), 3.59-3.56 (m, 1H), 3.34 (s, 3H), 3.08-3.05 (m, 1H), 3.02-2.99 (m, 1H), 2.92-2.89 (m, 1H), 2.81-2.76 (m, 1H), 2.21-2.12 (m, 4H), 1.86-1.83 (m, 1H), 1.59-1.55 (m, 1H), 1.35-1.29 (m, 6H). MS (ESI) 752.2 (M⁺).

### EXAMPLE 11

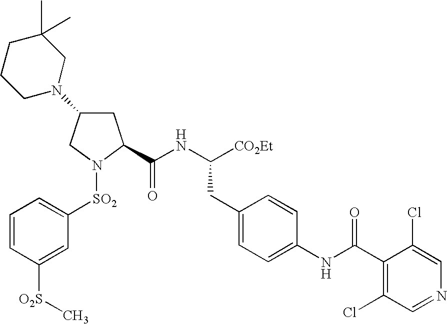

### N—{N-[(3-Methylsulfonylbenzene)sulfonyl]-4(R)-3,3-dimethylpiperidino-(L)-prolyl}-4-[(3′,5′-dichloro-isonicotinoyl)amino]-(L)-phenylalanine, Ethyl Ester

### Step A: N-(tert-butoxycarbonyl)-4(R)-3,3-dimethylpiperidino-L-proline, Methyl Ester

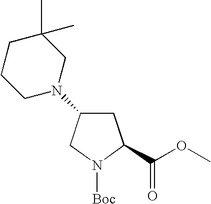

To a solution of N-(tert-butoxycarbonyl)-cis-hydroxy-L-proline methyl ester (1.5 g, 6.1 mmol) in CH₂Cl₂ was added diisopropylethylamine (2.7 mL, 16.1 mmol). After cooling the solution to −78° C., trifluoromethanesulfonic anhydride (1.4 mL, 8.3 mmol) was added dropwise over 10 min. After stirring for 1 h, the solution was warmed to −20° C. and 3,3-dimethylpiperidine (1.4 g, 12.4 mmol) was added dropwise over 5 min. The reaction was warmed to room temperature and stirred overnight. Water was added and the reaction was extracted with CH₂Cl₂. The combined organic layer was dried over MgSO₄, filtered, concentrated, and purified by silica gel chromatography to give the product as an oil. ¹H-NMR (DMSO-d₆, 500 MHz) δ 4.39-4.29 (m, 1H), 3.80-3.76 (m, 1H), 3.74 (s, 3H), 3.22-3.15 (m, 1H), 2.88-2.84 (m, 1H), 2.37-2.22 (m, 2H), 2.17-1.96 (m, 4H), 1.65-1.55 (m, 2H), 1.48-1.43 (2 s, 9H), 1.21-1.18 (m, 2H), 0.92 (s, 3H), 0.90 (s, 3H). MS (ESI) 341.3 (MH⁺).

### Step B: N—[N-(tert-butoxycarbonyl)-4(R)-3,3-dimethylpiperidino-(L)-prolyl]-4-[(3′,5′-dichloro-isonicotinoyl)amino]-(L)-phenylalanine, Ethyl Ester

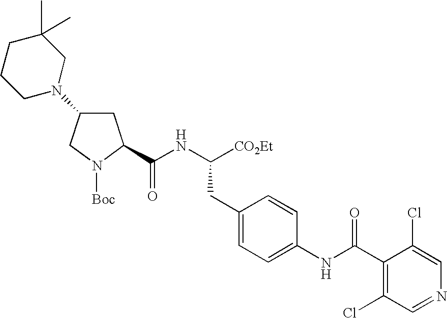

To a solution of N-(tert-butoxycarbonyl)-4(R)-3,3-dimethylpiperidino-L-proline methyl ester (1.22 g, 3.6 mmol) in acetonitrile/water (6 mL, 2/1) was added lithium hydroxide monohydrate (376 mg, 9.0 mmol). After stirring at room temperature for 1 h, the reaction was neutralized by the addition of 1N HCl (9 mL, 9 mmol) and lyophilized to provide the carboxylic acid which was used directly in the next step. MS (ESI) 327.3 (MH⁺).

To a suspension of the above acid in DMF (20 mL) was added N-methyl morpholine (1.0 mL, 9.1 mmol), HATU (1.5 g, 3.9 mmol), and 4-[(3′,5′-dichloroisonicotinoyl)amino]-(L)-phenylalanine ethyl ester hydrochloride (1.5 mg, 3.6 mmol). After stirring at room temperature for 2 h, the reaction was slowly added to water (200 mL) and the resulting precipitate was filtered and dried. The crude material was taken directly to the next step. MS (ESI) 690.3 (M⁺).

### Step C: N—{N-[(3-Methylsulfonylbenzene)sulfonyl]-4(R)-3,3-dimethylpiperidino-(L)-prolyl}-4-[(3′,5′-dichloro-isonicotinoyl)amino]-(L)-phenylalanine, Ethyl Ester

To a solution of N—[N-(tert-butoxycarbonyl)-4(R)-3,3-dimethylpiperidino-(L)-prolyl]-4-[(3′,5′-dichloro-isonicotinoyl)amino]-(L)-phenylalanine, ethyl ester (1.5 g, 2.2 mmol) in CH₂Cl₂ (20 mL) was added trifluoroacetic acid (5 mL). After stirring for 2 h at room temperature, the solution was concentrated and further dried under high vacuum. The crude material was dissolved in CH₂Cl₂ (10 mL) and triethylamine (0.65 mL, 4.7 mmol) followed by 3-methylsulfonylbenzenesulfonyl chloride (650 mg, 2.55 mmol) were added. After stirring for 1 h at room temperature, the reaction was concentrated and dried under vacuum. The crude material was dissolved in DMF (10 mL) and slowly added to water (100 mL). The precipitate was filtered, dissolved in CH₂Cl₂, dried over MgSO₄, filtered and concentrated. A portion of the product was taken to the next step without further purification, while the remainder was purified by preparative reversed-phase HPLC. The product fractions were lyophilized to give the title compound as a white powder. ¹H-NMR (DMSO-d₆, 500 MHz) δ 10.77 (s, 1H), 8.71 (s, 2H), 8.37 (d, 1H), 8.19-8.17 (m, 2H), 8.06-8.04 (m, 1H), 7.83-7.80 (m, 1H), 7.51 (d, 2H), 7.20 (d, 2H), 4.41-4.39 (m, 1H), 4.32-4.30 (m, 1H), 4.04-3.99 (m, 2H), 3.52-3.49 (m, 1H), 3.26 (s, 3H), 2.98-2.95 (m, 2H), 2.87-2.84 (m, 1H), 2.76-2.73 (m, 1H), 2.11-2.02 (m, 2H), 1.78-1.74 (m, 3H), 1.56-1.52 (m, 1H), 1.34-1.30 (m, 2H), 1.10 (t, 3H), 1.05-1.03 (m, 2H), 0.74 (s, 3H), 0.72 (s, 3H). MS (ESI) 808.2 (M⁺).

### EXAMPLE 12

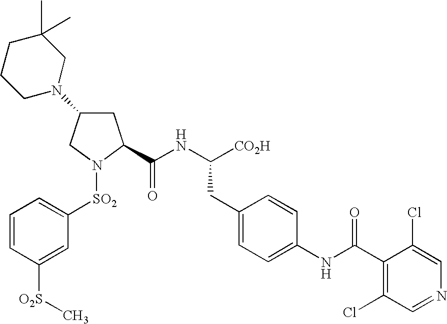

### N—{N-[(3-Methylsulfonylbenzene)sulfonyl]-4(R)-3,3-dimethylpiperidino-(L)-prolyl}-4-[(3′,5′-dichloro-isonicotinoyl)amino]-(L)-phenylalanine

To a solution of N—{N-[(3-methylsulfonylbenzene)sulfonyl]-4(R)-3,3-dimethylpiperidino-(L)-prolyl}-4-[(3′,5′-dichloro-isonicotinoyl)amino]-(L)-phenylalanine ethyl ester (Compound of Example 11, 160 mg, 0.20 mmol) in acetonitrile/water (4.5 mL, 2/1) was added lithium hydroxide monohydrate (20 mg, 0.48 mmol). After stirring at room temperature for 1 h, the reaction was quenched with 5 drops of formic acid. The reaction was directly purified by preparative reversed-phase HPLC and the product fractions were lyophilized to provide the title compound as a white powder. ¹H-NMR (DMSO-d₆, 500 MHz) δ 12.83 (s, 1H), 10.83 (s, 1H), 8.78 (s, 2H), 8.26-8.23 (m, 3H), 8.13-8.11 (m, 1H), 7.89-7.86 (m, 1H), 7.59-7.57 (d, 2H), 7.28-7.26 (d, 2H), 4.46-4.43 (m, 1H), 4.41-4.38 (m, 1H), 3.58-3.55 (m, 1H), 3.33 (s, 3H), 3.08-3.05 (m, 1H), 3.02-2.99 (m, 1H), 2.93-2.89 (m, 1H), 2.81-2.79 (m, 1H), 2.15-2.09 (m, 2H), 1.87-1.84 (m, 3H), 1.58-1.55 (m, 1H), 1.39-1.36 (m, 2H), 1.11-1.09 (m, 2H), 0.80 (s, 3H), 0.78 (s, 3H). MS (ESI) 780.2 (M⁺).

What is claimed is:

1. A compound of formula I: 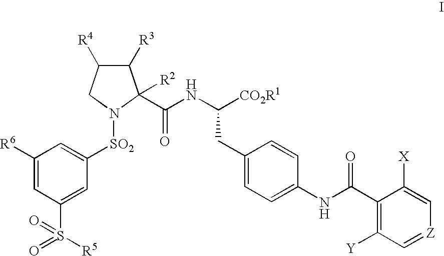
or a pharmaceutically acceptable salt thereof, wherein:
X and Y are independently chosen from (1) C₁₋₃alkyl, (2) halogen, and (3) C₁₋₃alkoxy;
Z is N or N⁺O⁻;
R¹ is selected from (1) hydrogen, (2) C₁₋₁₀alkyl, (3) —(C₁₋₁₀alkyl)-aryl, (4) —(C₁₋₁₀alkyl)-O—C₁₋₁₀alkyl, (5) —(C₁₋₁₀alkyl)-OC(O)—C₁₋₁₀alkyl, (6) —(C₁₋₁₀alkyl)-OC(O)-aryl, and (7) —(C₁₋₁₀ alkyl)-OC(O)O—C₁₋₁₀alkyl; wherein alkyl is optionally substituted with one to three substituents independently selected from R^{a} and aryl is optionally substituted with one to three substituents independently selected from Rb;
R² is hydrogen or methyl;
R³ and R⁴ are independently selected from (1) hydrogen, (2) C₁₋₁₀alkyl, (3) —OR^{d}, (4) —NR^{d}R^{e}, (5) —NR^{d}S(O)ₘR^{e}, (6) —NR^{d}C(O)R^{e}, (7) —NR^{d}C(O)OR^{e} and (8) —NR^{d}C(O)NR^{d}R^{e}, wherein alkyl is optionally substituted with one to four substituents independently selected from R^{a}; or
R³ and R⁴ together with the carbon atoms to which they are attached form a monocyclic ring of 5 to 7 members containing 0-2 additional heteroatoms independently selected from O, S and N—R^{h}, said ring optionally substituted with one to four substituents independently selected from R^{c};
R⁵ is selected from (1) C₁₋₁₀alkyl, and (2) aryl;
R⁶ is selected from (1) hydrogen, (2) halogen, and (3) —OR^{d};
R^{a} is selected from (1) —OR^{d}, (2) —NR^{d}S(O)ₘR^{e}, (3) —NO₂, (4) halogen, (5) —S(O)ₘR^{d}, (6) —SR^{d}, (7) —S(O)₂OR^{d}, (8) —S(O)ₘNR^{d}R^{e}, (9) —NR^{d}R^{e}, (10) —O(CR^{f}R^{g})ₙNR^{d}R^{e}, (11) —C(O)R^{d}, (12) —CO₂R^{d}, (13) —CO₂(CR^{f}R^{g})ₙCONR^{d}R^{e}, (14) —OC(O)R^{d}, (15) —CN, (16) —C(O)NR^{d}R^{e}, (17) —NR^{d}C(O)R^{e}, (18) —OC(O)NR^{d}R^{e}, (19) —NR^{d}C(O)OR^{e}, (20) —NR^{d}C(O)NR^{d}R^{e}, (21) —CR^{d}(N—OR^{e}), (22) CF₃, (23) —OCF₃, (24) C₃₋₈cycloalkyl, and (25) heterocyclyl; wherein cycloalkyl and heterocyclyl are optionally substituted with one to three groups independently selected from R^{c};
R^{b} is selected from (1) a group selected from R^{a}, (2) C₁₋₁₀ alkyl, (3) C₂₋₁₀ alkenyl (4) C₂₋₁₀ alkynyl, (5) aryl, and (6) —(C₁₋₁₀alkyl)-aryl, wherein alkyl, alkenyl, alkynyl, and aryl are optionally substituted with one to three substituents selected from a group independently selected from R^{c};
R^{c} is (1) halogen, (2) amino, (3) carboxy, (4) C₁₋₄alkyl, (5) C₁₋₄alkoxy, (6) aryl, (7) —(C₁₋₄alkyl)-aryl, (8) hydroxy, (9) CF₃, (10) OC(O)C₁₋₄alkyl, (11) —CN, and (12) —SO₂C₁₋₁₀alkyl;
R^{d} and R^{e} are independently selected from hydrogen, C₁₋₁₀ alkyl, C₂₋₁₀alkenyl, C₂₋₁₀alkynyl, Cy and Cy-C₁₋₁₀alkyl, wherein alkyl, alkenyl, alkynyl and Cy are optionally substituted with one to four substituents independently selected from R^{c}; or
R^{d} and R^{e} together with the atom(s) to which they are attached form a heterocyclic ring of 4 to 7 members containing 0-2 additional heteroatoms independently selected from O, S and N—R^{h}; wherein said ring is optionally substituted with one to four substituents independently selected from R^{c};
R^{f} and R^{g} are independently selected from hydrogen, C₁₋₁₀ alkyl, Cy and Cy-C₁₋₁₀alkyl; or
R^{f} and R^{g} together with the carbon to which they are attached form a ring of 5 to 7 members containing 0-2 heteroatoms independently selected from oxygen, sulfur and nitrogen;
R^{h} is selected from R^{f} and —C(O)R^{f};
Cy is selected from cycloalkyl, heterocyclyl, aryl, and heteroaryl;
each m is independently 0, 1 or 2; and
each n is independently 1, 2, 3, or 4.

2. The compound according to claim 1 wherein one of X and Y is halogen and the other is selected from halogen, C₁₋₃alkyl and C₁₋₃alkoxy.

3. The compound according to claim 2 wherein one of X and Y is chloro and the other is chloro or methoxy.

4. The compound according to claim 3 wherein X and Y are each chloro.

5. The compound according to claim 1 wherein R¹ is selected from the group consisting of: hydrogen, C₁₋₄alkyl, —(C₁₋₄alkyl)OC(O)—C₁₋₄alkyl, and —(C₁₋₄alkyl)OC(O)—C₁₋₄alkyl.

6. The compound according to claim 1 wherein R¹ is hydrogen.

7. The compound according to claim 1 wherein R¹ is C₁₋₄alkyl.

8. The compound according to claim 1 wherein R³ is hydrogen and R⁴ is NR^{d}R^{e}.

9. The compound according to claim 1 wherein R³ is NR^{d}R^{e} and R⁴ is hydrogen.

10. The compound according to claim 1 of formula Ia: 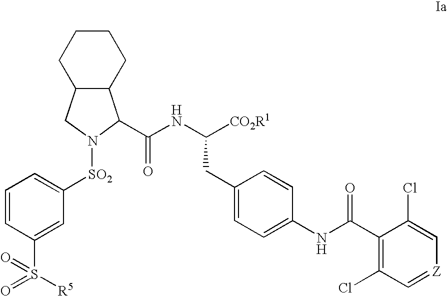
or a pharmaceutically acceptable salt thereof, wherein
Z is N or N⁺O⁻;
R¹ is selected from hydrogen, C₁₋₁₀alkyl, —(C₁₋₄alkyl)-aryl, —(C₁₋₄alkyl)-O—C₁₋₄alkyl, and —(C₁₋₄alkyl)-OC(O)—C₁₋₄alkyl; and
R⁵ is selected from C₁₋₄alkyl and phenyl.

11. The compound according to claim 1 selected from the following: 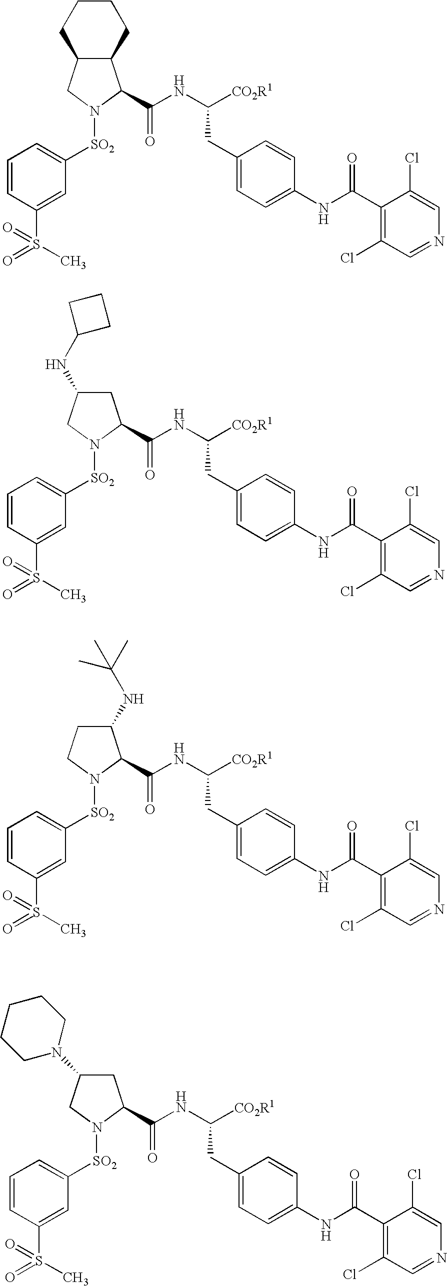
where R¹ is H or ethyl, and pharmaceutically acceptable salts thereof.

12. The compound according to claim 1 of Formula Ib: 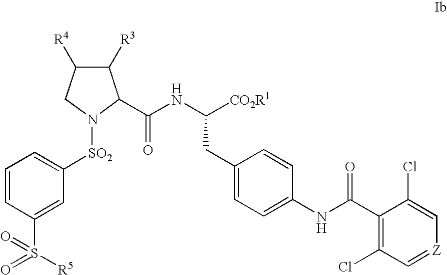
or a pharmaceutically acceptable salt thereof, wherein
Z is N or N⁺O⁻;
R¹ is selected hydrogen and C₁₋₄alkyl;
R⁵ is selected from C₁₋₄alkyl and phenyl; and
R³ is hydrogen and R⁴ is NR^{d}R^{e} or R³ is NR^{d}R^{e} and R⁴ is hydrogen.

13. The compound according to claim 12 wherein one of R³ or R⁴ is hydrogen and the other of R³ or R⁴ is selected from the group consisting of: C₁₋₆alkylamino, C₃₋₆cycloalkylamino and 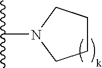
wherein k is 0 to 3.

14. The compound according to claim 13 wherein: one of R³ or R⁴ is hydrogen and the other of R³ or R⁴ is selected from the group consisting of: cyclobutylamino, tert-butylamino and piperidino.

15. A compound according to claim 1 selected from the group consisting of:
(R,S,S)—N—{N-[(3-Methylsulfonylbenzene)sulfonyl]-4(R)-cyclobutylamino-(L)-prolyl}-4-[(3,5′-dichloro-isonicotinoyl)amino]-(L)-phenylalanine, ethyl ester;
(R,S,S)—N—{N-[(3-Methylsulfonylbenzene)sulfonyl]-4(R)-cyclobutylamino-(L)-prolyl}-4-[(3′,5′-dichloro-isonicotinoyl)amino]-(L)-phenylalanine;
N—{N-[(3-Methylsulfonylbenzene)sulfonyl]-3(−)-tert-butylamino-(L)-prolyl}-4-[(3′,5′-dichloro-isonicotinoyl)amino]-(L)-phenylalanine, ethyl ester;
N—{N-[(3-Methylsulfonylbenzene)sulfonyl]-3-tert-butylaminoprolyl}-4-[(3′,5′-dichloro-isonicotinoyl)amino]-(L)-phenylalanine;
(S,R,S,S)—N—{N-[(3-Methylsulfonylbenzene)sulfonyl]octahydroisoindol-1-carboxyl}-4-[(3′,5′-dichloro-isonicotinoyl)amino]-(L)-phenylalanine, ethyl ester;
(S,R,S,S)—N—{N-[(3-Methylsulfonylbenzene)sulfonyl]octahydroisoindol-1-carboxyl}-4-[(3′,5′-dichloro-isonicotinoyl)amino]-(L)-phenylalanine;
(S,R,S,S)—N—{N-[(3-Phenylsulfonylbenzene)sulfonyl]octahydroisoindol-1-carboxyl}-4-[(3′,5′-dichloro-isonicotinoyl)amino]-(L)-phenylalanine, ethyl ester;
(S,R,S,S)—N—{N-[(3-Phenylsulfonylbenzene)sulfonyl]octahydroisoindol-1-carboxyl}-4-[(3′,5′-dichloro-isonicotinoyl)amino]-(L)-phenylalanine;
N—{N-[(3-Methylsulfonylbenzene)sulfonyl]-4(R)-piperidino-(L)-prolyl}-4-[(3′,5′-dichloro-isonicotinoyl)amino]-(L)-phenylalanine, ethyl ester;
N—{N-[(3-Methylsulfonylbenzene)sulfonyl]-4(R)-piperidino-(L)-prolyl}-4-[(3′,5′-dichloro-isonicotinoyl)amino]-(L)-phenylalanine;
N—{N-[(3-Methylsulfonylbenzene)sulfonyl]-4(R)-3,3-dimethylpiperidino-(L)-prolyl}-4-[(3′,5′-dichloro-isonicotinoyl)amino]-(L)-phenylalanine, ethyl ester; and
N—{N-[(3-Methylsulfonylbenzene)sulfonyl]-4(R)-3,3-dimethylpiperidino-(L)-prolyl}-4-[(3′,5′-dichloro-isonicotinoyl)amino]-(L)-phenylalanine,
or a pharmaceutically acceptable salt of any of the above.

16. A pharmaceutical composition comprising a therapeutically effective amount of a compound of claim 1 or a pharmaceutically acceptable salt thereof and a pharmaceutically acceptable carrier.

17. Use of a compound of claim 1 or a pharmaceutically acceptable salt thereof for the manufacture of a medicament for the treatment or prevention of diseases mediated by cell adhesion.

18. The use of claim 17 wherein said disease is selected from asthma, multiple sclerosis, inflammatory bowel disease, chronic obstructory pulmonary disease, sickle cell anemia, leukemia, multiple myeloma, and rheumatoid arthritis.

19. A method for preventing the action of VLA-4 in a mammal which comprises administering to said mammal a therapeutically effective amount of a compound according to claim 1.
